(19) United States
(12) Patent Application Publication
(10) Pub. No.: US 2022/0167490 A1
(43) Pub. Date: May 26, 2022

(54) METHOD AND APPARATUS FOR ENSURING AND TRACKING ELECTROSTATIC DISCHARGE SAFETY AND COMPLIANCE

(71) Applicant: William C. Berg, Lakeville, MN (US)

(72) Inventors: William C. Berg, Lakeville, MN (US); Matthew J. Loegering, Edina, MN (US)

(21) Appl. No.: 17/442,124

(22) PCT Filed: Mar. 24, 2020
(86) PCT No.: PCT/US2020/024517

§ 371 Date: Sep. 22, 2021

Related U.S. Application Data
(60) Provisional application No. 62/823,856, filed on Mar. 26, 2019.

(51) Int. Cl. H05F 3/02; G01R 19/165

(52) U.S. Cl. CPC H05F 3/02; G01R 19/16533

(57) ABSTRACT

A method and apparatus for ensuring and tracking electrostatic discharge (ESD) safety and compliance via electronics that gather information and parameters from wearable electronics, such as a wrist strap, worn by each user at a company's electronics-manufacturing plant or similar facility that indicate whether and when a functional ESD ground is established and continually maintained between each respective user and each workstation at which each respective user locates, wherein a system monitor is configured to receive on a continual or periodic basis whether each user is complying with the ESD policy of the company. In some embodiments, the parts worked on at each workstation, billing and job numbers, and/or employee work hours are also tracked by a computer server that aggregates data from one or more system monitors and generates compliance reports for each part, job, and/or employee.

### CROSS-REFERENCE TO RELATED APPLICATION

This application claims priority benefit, including under 35 U.S.C. § 119(e), of U.S. Provisional Patent Application 62/823,856 filed Mar. 26, 2019 by William C. Berg, et al., titled “System, apparatus, and method to ensure electrostatic discharge safety compliance,” which is incorporated herein by reference in its entirety.

### FIELD OF THE INVENTION

This invention relates to the field of electronics, and more specifically to a system, method and apparatus for ensuring and tracking electrostatic discharge (ESD) safety and compliance via electronics, such as a wearable wrist strap worn by each user and ESD safety-related equipment used at a company's electronics manufacturing plant or similar facility, wherein the electronics gathers information and parameters that indicate whether a functional ESD ground is established and continually maintained between each user and a workstation at which the user is located, wherein a central location is configured to receive information on a continual or periodic basis whether each user is complying with the ESD policy of the company.

### BACKGROUND OF THE INVENTION

Many electronic components are highly susceptible to damage by electrostatic discharge (ESD) from people who handle the components, or who handle assemblies containing the components. A common method of preventing ESD damage is to have people wear a conductive strap around a wrist or ankle which contacts the person's skin, and then connect that strap to earth ground with a conductive wire. A person so connected is said to be grounded. In manufacturing environments, it is very important and very difficult to ensure that personnel are always grounded when handling sensitive electronic components and assemblies. The more personnel who have to be monitored, the more difficult it becomes to detect negligent personnel. In high-reliability applications where ESD damage cannot be tolerated, intensive monitoring is required, which is both expensive and time-consuming. Many conventional ESD-protection compliance schemes focus on the workstation and ESD discharge mats at the workstation. Often, those schemes fail at some point because the users do not realize that their ESD-protection connections have been interrupted, disconnected, or the like. These schemes do not continuously monitor each worker and assess his or her ESD-safety status.

ESD policies are set by companies to prevent losses due to electrostatic discharge from persons to electronic components with which the person may be working. Companies try to ensure that employees and others who may come into contact with sensitive electronic components comply with each company's ESD protection policy.

Most conventional ESD-monitoring systems only provide an alarm if a conductive strap is not connected to a monitor which is mounted at a work station; no record is kept of connect or disconnect events, nor of the identity of the person who is working at the station.

U.S. Pat. No. 9,291,661 to Liu issued Mar. 22, 2016 with the title “Monitoring circuit and system for ESD protection device,” and is incorporated herein by reference. U.S. Pat. No. 9,291,661 describes a specific circuit to monitor the connection to ground of an ESD device worn by a user. The monitoring circuit includes an oscillating unit, a signal processing unit and a comparator. The oscillating unit includes a first monitoring end and a second monitoring end. The first monitoring end is configured to be electrically connected to an ESD protective device. The second monitoring end is configured to be electrically connected to ground. When the first monitoring end is not electrically contacted to a user's body or the second monitoring end is not connected to ground, the oscillating unit is configured to output an oscillating signal. The signal processing unit is electrically connected to the oscillating unit, and is configured to output a first voltage according to the oscillating signal. The comparator is configured to compare the first voltage and a reference voltage, and correspondingly output an alarm signal.

U.S. Pat. No. 6,205,408 to Jubin et al. issued Mar. 20, 2001 with the title “Continuous monitoring system,” and is incorporated herein by reference. U.S. Pat. No. 6,205,408 describes automated systems for performing electrostatic discharge (ESD) device efficacy monitoring and recording the results for an ESD auditing program. Systems of U.S. Pat. No. 6,205,408 include at least one ESD device monitoring unit. A communication system allows the monitoring unit to communicate with a central computer which collects, stores and allows the manipulation of the test data. Systems of U.S. Pat. No. 6,205,408 are therefore useful in testing the ESD devices, documenting their performance, and controlling access to particular work areas based on testing results.

U.S. Pat. No. 4,638,399 Maroney, et al. issued Jan. 20, 1987 with the title “Wrist strap ground monitor,” and is incorporated herein by reference. U.S. Pat. No. 4,638,399 describes an apparatus which can be embodied in an electronic wristwatch monitors the integrity of a wrist strap ground. An input terminal to which a known ground is coupled is provided. An oscillator produces a fixed frequency which is mixed with a signal from the input terminal to provide a composite signal. The composite signal is coupled to one input of an exclusive OR-gate. The other input of the exclusive OR-gate is coupled directly to the output of the oscillator. The output of the OR-gate is processed to produce an output signal indicative of the phase relationship between the oscillator output and the composite signal. When the input terminal is grounded, the phase relationship between the oscillator output and the composite signal changes, resulting in a change in the output signal which can be used to trigger an indicator (e.g., visual display and/or aural alarm) to indicate to a user whether he or she is properly grounded.

U.S. Pat. No. 3,774,106 to MacPhee issued Nov. 20, 1973 with the title “Electrical grounding system and ground integrity checker,” and is incorporated herein by reference. U.S. Pat. No. 3,774,106 describes an electrical grounding system for equipment (such as electrical instruments in an intensive-care hospital room) includes two ground conductors, both connected to a common ground and to the equipment to be grounded and forming a loop. In case of a break in one conductor between the equipment and ground, the other conductor maintains the equipment ground. A ground-integrity checking transformer has a secondary winding of few turns interposed as a series element in the grounding loop and injects only a minimal test voltage in the loop. A primary winding of many turns is used for impressing excitation; and the primary winding is in a test circuit that evidences a break in the grounding loop.

ESD policy enforcement and management are difficult because many conventional systems lack consistent and constant user accountability.

There remains a need in the art for an improved system for checking, alerting, and recording compliance with ESD rules for a workplace.

### SUMMARY OF THE INVENTION

The present invention provides a system, method and apparatus for tracking and monitoring each particular user's compliance to an electrostatic discharge (ESD) policy; ensuring and tracking ESD safety and compliance of each user (via a unique identifier (ID), such as a serial number associated with each user) via electronics that gather information and parameters from the wearable ESD device such as a wrist strap worn by each user at a company's electronics-manufacturing plant or similar facility that indicate whether a functional ESD ground is established and continually maintained between each user and a workstation at which the user is located, wherein an ESD data-collection system monitor at a central location is configured to receive information on a continual or periodic basis as to whether each user is complying with the ESD policy of the company.

In some embodiments, the present invention determines by direct measurement (such as measurement of the user's skin resistance, capacitance, and/or radio-frequency (RF) conduction, and/or heart rate, skin temperature or the like measured by, for example, a wrist-mounted sensor unit) whether or not the user is wearing the wearable ESD device. In other embodiments, the present invention determines whether the wearable ESD device is in contact with a user's skin by inference based on one or more parameters (such as tautness of a wrist strap, a switch closure, a pressure sensor, temperature-difference sensor, and/or machine-vision image analysis or the like) whether the user is wearing the wearable ESD device. In some embodiments, each connection and disconnection event of the wearable ESD device to each ESD interface unit at a respective workstation is recorded and timestamped to track compliance to criteria of an ESD compliance policy. In some embodiments, such recording and timestamps can be used for, e.g., workplace tracking of hourly job-related work activities. Some embodiments track which products were worked on (e.g., by tracking each part's serial number) by which workers at which workstations to facilitate such activities as product recalls and warranty costs or the like. In some embodiments, the present invention elicits and receives logins and logouts of users relative to jobs being performed and billed for, and such data can be analyzed for efficiency reports and employee evaluations, as well as compliance to ESD policies. Some embodiments determine whether some user is present at a workstation but not electrically connected to earth ground at the workstation, and set an alarm and/or record the occurrence of such events.

Some embodiments in which the wearable ESD device communicates wirelessly, determine user whereabouts by detecting whether the wearable ESD device is close enough to communicate with other compatible wireless units whose location is known. In some embodiments, such information regarding a user's whereabouts is optionally used to determine whether a user is following work instructions, adhering to schedule, or otherwise following company policy.

Some embodiments of the wearable ESD device include a camera, scanner or other sensor for scanning barcodes, an RF reader for reading RF-identification (RFID) tags, and/or other input device for reading identification data associated with production equipment or supplies, product components or assemblies, documentation, and the like. In some embodiments, such device(s) permit the user to indicate when a step in a production process has been started or completed; to record the location of equipment, supplies, components, assemblies, documentation, etc.; to take or release possession of (check-out or check-in) production equipment or supplies; or otherwise signal or record presence or absence of identified equipment, supplies, components, assemblies, documentation, and the like.

Some embodiments of the wearable ESD device include a camera with which a user may take pictures to obtain images that record defects in product assemblies, status of work-in-progress, or other information regarding products or processes in a manufacturing environment.

In some embodiments, the wearable ESD device includes circuitry and/or software to allow expanded communications between users and company-wide manufacturing software including Enterprise Resource Planning (ERP) and Computer Aided Manufacturing (CAM). In some embodiments, users are able to log in and out of jobs, determine a schedule, determine time standards, keep track of time, obtain instructions, solicit help, place material orders, and/or initiate alerts.

In some embodiments, the present invention permits continuous monitoring of a human operator's ESD-grounding state—i.e., individual compliance—and ESD-system-component integrity at all times, regardless of the operator's location in a manufacturing facility. The operator wears a wearable ESD device whenever the operator is on duty. In some embodiments, the wearable ESD device has a unique identification which is mapped to the operator, so any data collected and transmitted by the wearable ESD device are placed in the operator's record. In some embodiments, the wearable ESD device can detect whether it is in contact with the operator's skin, and whether it is connected by a conductor, such as a conductive wire, to a grounded work station.

In some embodiments, an indication of any change in the wearable ESD device's connection status, either the connection to the operator's skin or the connection to the work station, is transmitted along with the wearable ESD device's identification to an ESD data-collection system monitor which collects and stores the data. The system monitor may take immediate action if an operator is not grounded (by sounding an alarm, notifying a supervisor, etc.), or the data may be reviewed later as a means of auditing ESD grounding compliance.

In some embodiments, a system of the present invention includes: a first wearable ESD device configured to be worn by a user, wherein the first wearable ESD device includes: a machine-readable identification number associated with the first wearable ESD device; an electrical connection configured to be connected to a workstation that has a connection to an earth ground; an electrode that provides electrical conductivity between the first wearable ESD device and the user's skin; and a wearable-device communications circuit configured to transmit, to an ESD data-collection system monitor, a plurality of parameters including the identification number and an indication of an electrical connection between the user's skin and the earth ground at the work station.

In some embodiments, a system of the present invention alternatively or additionally includes: a first ESD interface device configured to be associated with the first work station, wherein the first interface device includes: a machine-readable identification number associated with the first ESD interface device; an electrical connection configured to be connected to the earth ground at the first work station; an electrical connection that provides electrical conductivity between the first ESD interface device and a first wearable ESD device, wherein the first wearable ESD device has a machine-readable identification number and is configured to provide electrical contact to the first user's skin; and an ESD-interface-device communications circuit configured to communicate, to an ESD data-collection system monitor, a plurality of parameters including: the identification number of the first wearable ESD device, the identification number of the first ESD interface device, and an indication of an electrical connection between the user's skin and the earth ground at the first work station.

In some embodiments, the present invention alternatively or additionally includes: an ESD data-collection system monitor that is programmed to elicit and receive communications from a first wearable ESD device configured to be worn by a user and/or communications from a first ESD interface device configured to be associated with the first work station, wherein the communications include an identification code associated with the first wearable ESD device and an identification code associated with the first ESD interface device, and programmed to record connection and disconnection events between the first wearable ESD device and the first ESD interface device and to record associated timestamps for each of the connection and disconnection events.

### BRIEF DESCRIPTION OF THE DRAWINGS

FIG. 1A is a block diagram of a basic ESD monitoring and compliance system 101, according to some embodiments of the present invention.

FIG. 1B is a block diagram of an ESD monitoring and compliance system 102 that includes a sensor unit 170 having strap switch 127, according to some embodiments of the present invention.

FIG. 2 is a block diagram of an expanded system 200, according to some embodiments of the present invention.

FIG. 3A is a perspective view and block diagram of a system 301 that includes a sensor unit 310 on the wrist 96 of a user 99, according to some embodiments of the present invention.

FIG. 3B is a data flow and management diagram of a system 302, according to some embodiments of the present invention.

FIG. 3C is a block diagram of a networked ESD compliance system 303, according to some embodiments of the present invention.

FIG. 3D is a block diagram of a cloud-based ESD compliance system 304, according to some embodiments of the present invention.

FIG. 3E is a block diagram of a system 305 using various connectivity methods to communicate with a system monitor 321, according to some embodiments of the present invention.

FIG. 3F is a block diagram of a system 306 using a cellular network to communicate with a system monitor 321, according to some embodiments of the present invention.

FIG. 4 includes a side view of conductive ESD strap assembly 401 and block diagram 402 of a system 400 that includes a sensor unit 110, according to some embodiments of the present invention.

FIG. 5 includes a side view of conductive ESD strap assembly 501 (with switch 127 open), side view 501′ (with switch 127 closed) and block diagram 502 of a system 500 that includes a sensor unit 170, according to some embodiments of the present invention.

FIG. 6 is a block diagram of a system 600 that includes a skin-resistance sensor unit 610, according to some embodiments of the present invention.

FIG. 7 is a block diagram of a system 700 that includes a wrist-switch sensor unit 710, according to some embodiments of the present invention.

FIG. 8 is a block diagram of a sensor-unit system 800 that includes a skin-resistance sensor unit 810, according to some embodiments of the present invention.

FIG. 9 is a block diagram of a system 900 that includes a skin-resistance sensor unit 910 with a single-conductor cable 920, according to some embodiments of the present invention.

FIG. 10A is a block diagram of a system 1001 that includes a skin-resistance sensor unit 1010, according to some embodiments of the present invention.

FIG. 10B is a block diagram of a system 1002 that includes a skin-resistance sensor unit 1010, according to some embodiments of the present invention.

FIG. 11A is a block diagram of a system 1101 that includes a skin-resistance sensor unit 1010, according to some embodiments of the present invention.

FIG. 11B is a block diagram of a system 1102 that includes a skin-resistance sensor unit 1010, according to some embodiments of the present invention.

FIG. 12 is a block diagram of a system 1200 that includes an interface unit 1230, according to some embodiments of the present invention.

FIG. 13A is a block diagram of a system 1301 that uses communications to a system monitor 321 via a sensor unit 1310, according to some embodiments of the present invention.

FIG. 13B is a block diagram of a system 1302 that uses communications to a system monitor 321 via an interface unit 1332, according to some embodiments of the present invention.

FIG. 13C is a block diagram of a system 1303 that uses communications to a system monitor 321 via a smartphone 1360 and a sensor unit 1313, according to some embodiments of the present invention.

FIG. 13D is a block diagram of a system 1304 that uses a proximity detector that detects whether there is a user at the workstation but not connected to the interface unit, according to some embodiments of the present invention.

FIG. 14 is a block diagram of a system 1400 that uses communications to a system monitor 321 via a sensor unit 1010, according to some embodiments of the present invention.

FIG. 15A is a schematic diagram of a sensor unit 1501 usable with various ones of the system embodiments described herein, according to some embodiments of the present invention.

FIG. 15B is a block diagram of a sensor unit circuit 1502 usable with various ones of the system embodiments described herein, according to some embodiments of the present invention.

FIG. 16 is a flowchart of a method 1600 usable with various ones of the sensor-unit embodiments described herein, according to some embodiments of the present invention.

FIG. 17A is a schematic diagram of an alternative sensor unit 1701 that uses LED status indicators, rather than the LCD used by sensor unit 1501 of FIG. 15A, according to some embodiments of the present invention.

FIG. 17B is a block diagram of a sensor unit circuit 1702 that uses LED status indicators, rather than the LCD used by sensor unit 1502 of FIG. 15B, according to some embodiments of the present invention.

FIG. 18A is a schematic diagram of an alternative sensor unit 1801 that uses LED status indicators and user-configuration switches, rather than the LCD, and a wrist-strap switch rather than skin-resistance sensing used by sensor unit 1501 of FIG. 15A, according to some embodiments of the present invention.

FIG. 18B is a block diagram of a sensor unit circuit 1802 that uses LED status indicators, rather than the LCD, and a wrist-strap switch rather than skin-resistance sensing, used by sensor unit 1502 of FIG. 15B, according to some embodiments of the present invention.

### COPYRIGHT NOTICE/PERMISSION

A portion of the disclosure of this patent document contains material which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in the attached figures: Copyright© 2018-2020, William C. Berg, All Rights Reserved.

### DESCRIPTION OF PREFERRED EMBODIMENTS

Although the following detailed description contains many specifics for the purpose of illustration, a person of ordinary skill in the art will appreciate that many variations and alterations to the following details are within the scope of the invention. Specific examples are used to illustrate particular embodiments; however, the invention described in the claims is not intended to be limited to only these examples, but rather includes the full scope of the attached claims. Accordingly, the following preferred embodiments of the invention are set forth without any loss of generality to, and without imposing limitations upon the claimed invention. Further, in the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The embodiments shown in the Figures and described here may include features that are not included in all specific embodiments. A particular embodiment may include only a subset of all of the features described, or a particular embodiment may include all of the features described.

The leading digit(s) of reference numbers appearing in the Figures generally corresponds to the Figure number in which that component is first introduced, such that the same reference number is used throughout to refer to an identical component which appears in multiple Figures. Signals and connections may be referred to by the same reference number or label, and the actual meaning will be clear from its use in the context of the description.

Certain marks referenced herein may be common-law or registered trademarks of third parties affiliated or unaffiliated with the applicant or the assignee. Use of these marks is for providing an enabling disclosure by way of example and shall not be construed to limit the scope of the claimed subject matter to material associated with such marks.

Overview

More sophisticated conventional ESD monitoring systems, such as that described in U.S. Pat. No. 6,205,408 (which is incorporated herein by reference), do keep records of connect and disconnect events at a station, but can only determine the person's identity by inference from a data table which indicates (assumes) which person is supposed to be at the station. Furthermore, personnel are assumed to ground themselves when at a workstation, but personnel who fail to ground themselves cannot be detected or identified. These two assumptions seldom hold true.

Electrostatic discharge (ESD) into electronic components or assemblies can cause damage or failure of the parts and systems into which the parts are incorporated. Individuals trained to handle, assemble, or manufacture electronic components or assemblies (electronics) will employ strategies to protect the electronics. Companies often have ESD policies mandating methods and procedures to be followed by their personnel to minimize ESD damage. A current and common practice to prevent ESD damage requires individuals handling ESD-sensitive items to attach a conductive strap to his or her skin at the wrist and/or ankle. These straps will connect to an ESD ground or earth ground with a wire or other electrical conductor. Such conductors discharge static electrical charge and prevent accumulated charge. Conventional ESD-prevention methods often require multiple actions performed at multiple separate locations. The first action is to fasten a strap to the skin on one's wrist or ankle. The second action is to measure the integrity of the installed strap connection. This is typically done with dedicated equipment at a standalone station. The dedicated equipment's specific purpose is to measure and validate a user's skin-to-strap and strap-to-conductive-cable ESD connection. The third action involves recording data from the measurement. In the fourth action, the worker will leave the dedicated equipment station, go to a work area, and connect himself or herself to an ESD-grounded work station with the conductive cable. While this method for ESD prevention is often mandated (e.g., by company policy), compliance to the policy and connection integrity often depends on individual awareness and discipline. Existing conventional ESD-compliance systems monitor connection to the work area or work station. Existing systems do not identify each user or differentiate users who may occasionally be connected to work areas. Existing systems do not monitor or account for continuous worker compliance. The present invention helps ensure that workers will maintain ESD protection and compliance by directly and continuously monitoring the worker's strap connection to his or her skin and/or to an ESD-grounded work area. The present invention also records and tracks user-ESD and/or equipment-ESD connectivity throughout the work shift. In some embodiments, each wearable ESD device has a unique and permanent identification number (ID). With a unique identifier (ID) assigned to each wearable ESD device (or, in some embodiments, the number is relatively unique so as to be able to track each user in a facility as separately identifiable individuals), the system can track each worker's ESD status throughout the work day and/or across an inventory of parts and/or work stations on which the worker worked. In some embodiments, the wearable ESD device verifies the user's skin connection to the wearable ESD device and determines the worker's ESD status. In some embodiments, the interface device that connects the user's wearable ESD device to an ESD ground is also assigned a permanent and unique ID. By system-level polling of each worker's wearable ESD device, one can determine the worker's ESD status, compliance, and location. A “safe status” occurs when the strap is properly worn by the worker and connected to a safe ESD connection. Companies manufacturing high-reliability electronics components and assemblies are often mandated to follow ESD-control-program standards, such as the ANSI/ESD 520.20, and are audited to such standards. Continual monitoring of user and equipment compliance by the management of the company is very difficult, if not impossible, using conventional methods and equipment. The present invention saves additional set-up or indirect work time (and the associated worker wages) related to validating worker's wrist-strap connections at dedicated equipment, facilitates and save time related to manager or administrative ESD-compliance program requirements, and provides high confidence program records of worker- and system-ESD-compliance.

System Description

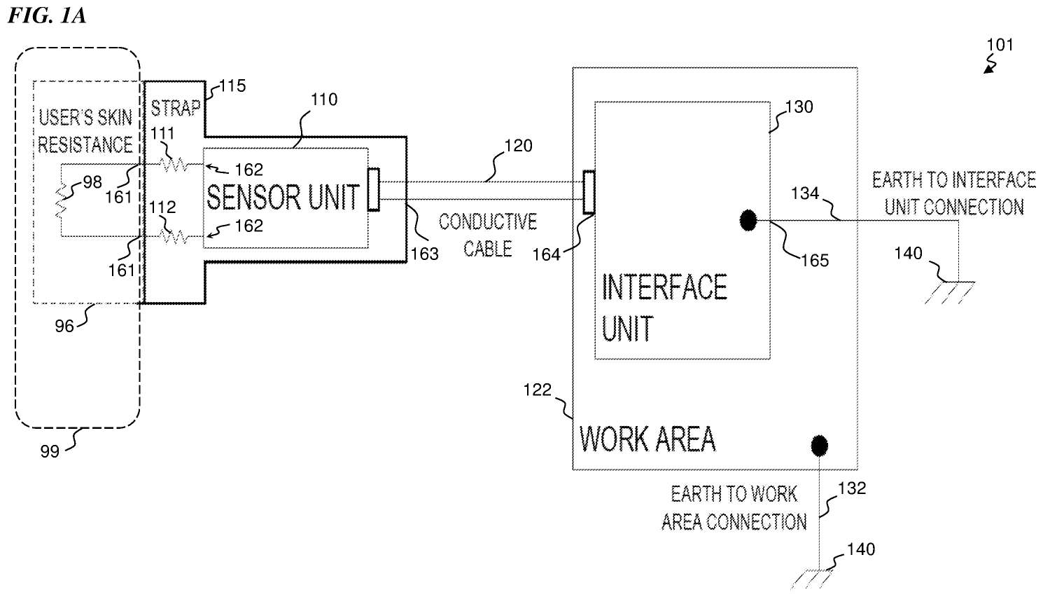

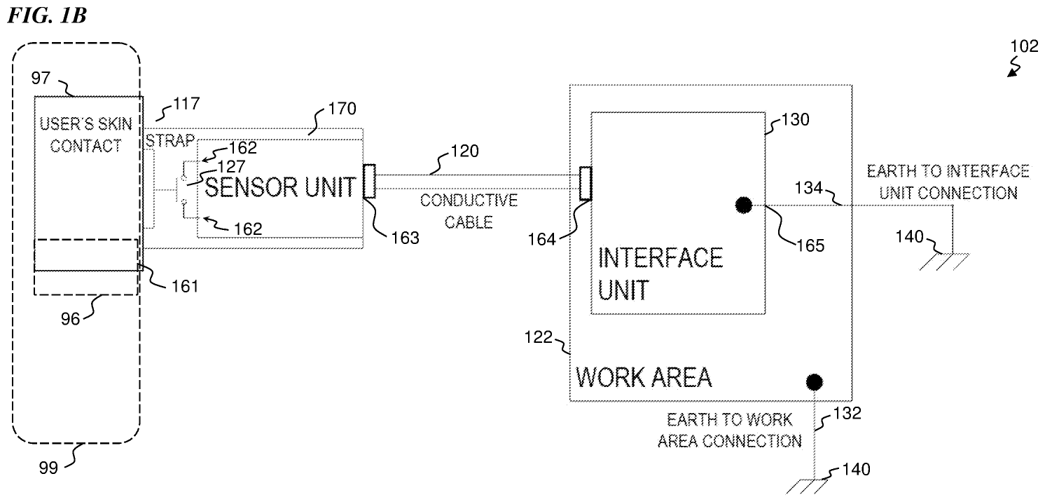

FIG. 1A is a block diagram of a basic ESD monitoring and compliance system 101, according to some embodiments of the present invention. In some embodiments, system 101 includes a sensor unit 110 held to the wrist 96 of a user 99 by a strap 115. In some embodiments, strap 115 includes inherent resistance 111 and resistance 112 that are in series with skin resistance 98. Sensor unit 110 is configured to measure the skin resistance 98 of the user 99 at her or his wrist 96. In some embodiments, sensor unit 110 is connected via conductive cable 120 to an interface unit 130 at a work area 122. In some embodiments, interface unit 130 is connected to earth ground 140 via a conductor 134. In some embodiments, work area 122 is connected to earth ground 140 via a conductor 132.

FIG. 1B is a block diagram of an ESD monitoring and compliance system 102 that includes a sensor unit 170 having strap switch 127, according to some embodiments of the present invention. In some embodiments, system 102 includes a sensor unit 170 held to the wrist of a user 99 by a strap 117. Sensor unit 170 is configured to determine whether user 99 is connected to sensor unit 170 by sensing the open/closed state of switch 127. Sensor unit 170 is connected via conductive cable 120 to an interface unit 130 at a work area 122. In some embodiments, interface unit 130 is connected to earth ground 140 via a conductor 134. In some embodiments, work area 122 is connected to earth ground 140 via a conductor 132.

In some embodiments, there are five connection points between a user's skin and an ESD or earth ground (as shown in FIG. 1A and FIG. 1B). The five connection points are: connection point 161 between the user's skin 96 and conductive strap 115 of sensor unit 110 or conductive strap 117 of sensor unit 170, connection point 162 between the conductive strap 115 and its sensor unit 110 or between conductive strap 117 and its sensor unit 170, connection point 163 between the sensor unit 110 or 170 and the conductive cable 120, connection point 164 between the conductive cable 120 and the interface unit 130, and connection point 165 between the interface unit 130 and ESD or earth ground 140. In many cases, users work on work area surfaces 122 also requiring an ESD or earth grounded connection 134. A system of the present invention in its most generic or basic embodiment would include a sensor unit 110 or sensor unit 170, a conductive cable 120, and a basic interface unit 130. A basic interface unit 130 electrically connects the conductive cable 120 to an ESD or earth ground 140.

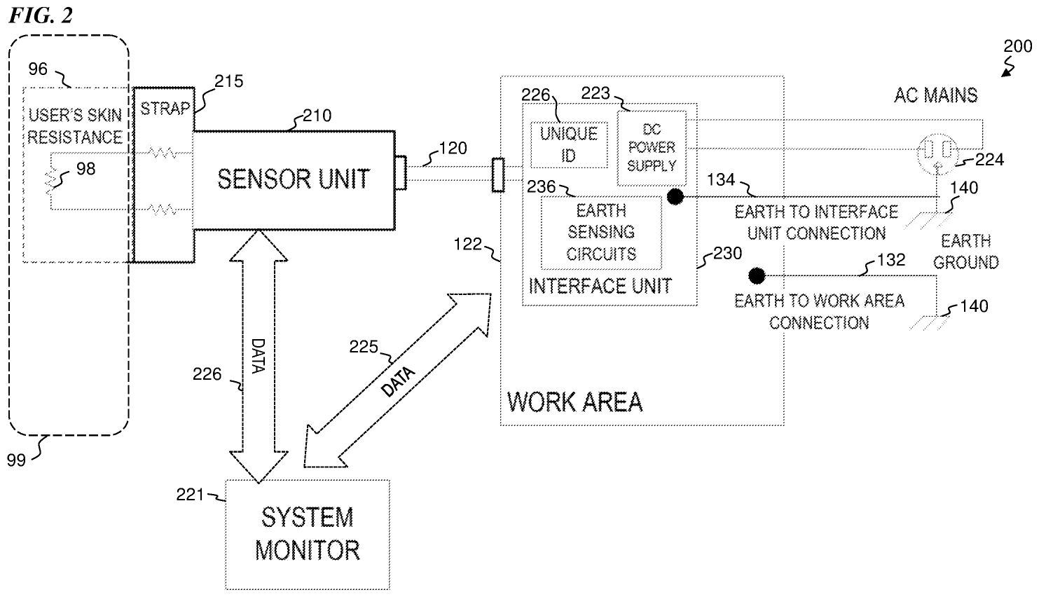

FIG. 2 is a block diagram of an expanded system 200, according to some embodiments of the present invention. In some embodiments, expanded system 200 in this preferred embodiment includes an interface unit 230 with additional functionality and a system monitor 221. In some embodiments, expanded system 200 provides methods and systems for validating, measuring, and/or monitoring connectivity between a user's skin 96 and an ESD wearable ESD device 210 with a conductive wrist strap 215, and validating, measuring and/or monitoring connection to an ESD or earth ground 140. Additionally, expanded system 200 provides methods and systems for validating, measuring, and/or monitoring connectivity between a user's work surface 122 and ESD or earth ground 140. In some embodiments, expanded system 200 also provides methods and systems for displaying, annunciating (such as by a speaker or buzzer) and/or logging user-connectivity status. By including a permanent and unique identifier (ID) assigned to and/or included in each ESD system device (e.g., each wrist strap, interface unit, work area and the like), the methods and systems can track and differentiate users and use locations. With the use of system monitor 221, the present invention also provides methods and systems to collect, manage, and/or report multiple user ESD-connectivity data and/or to communicate with users.

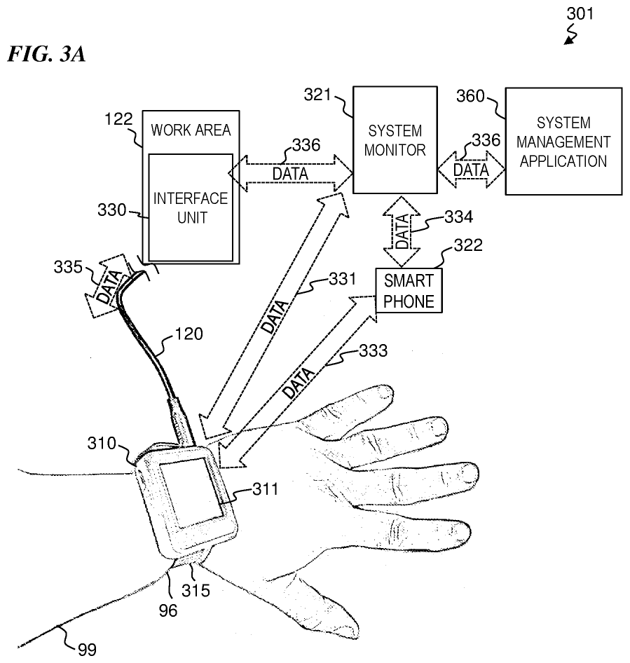

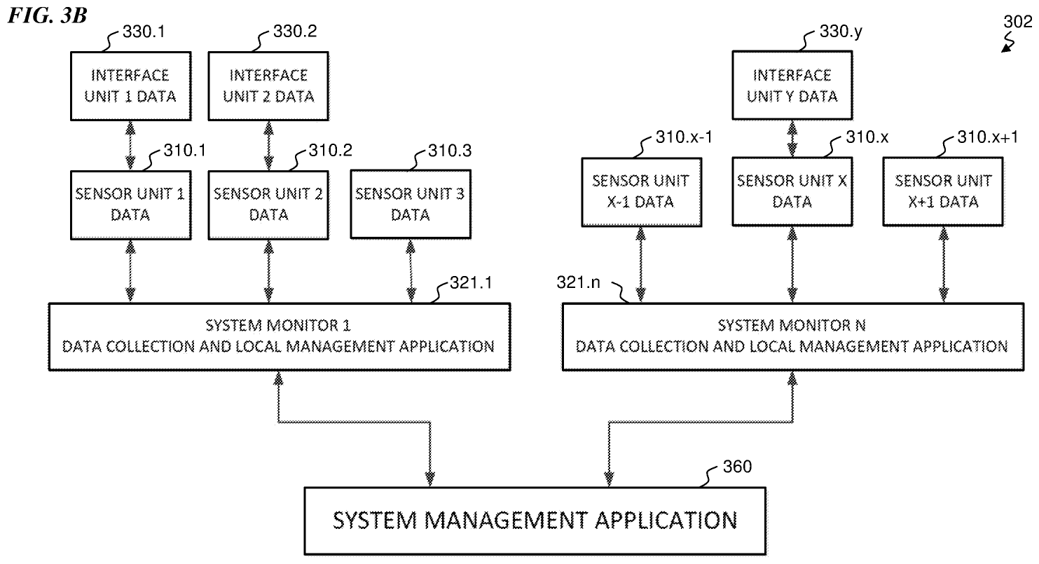

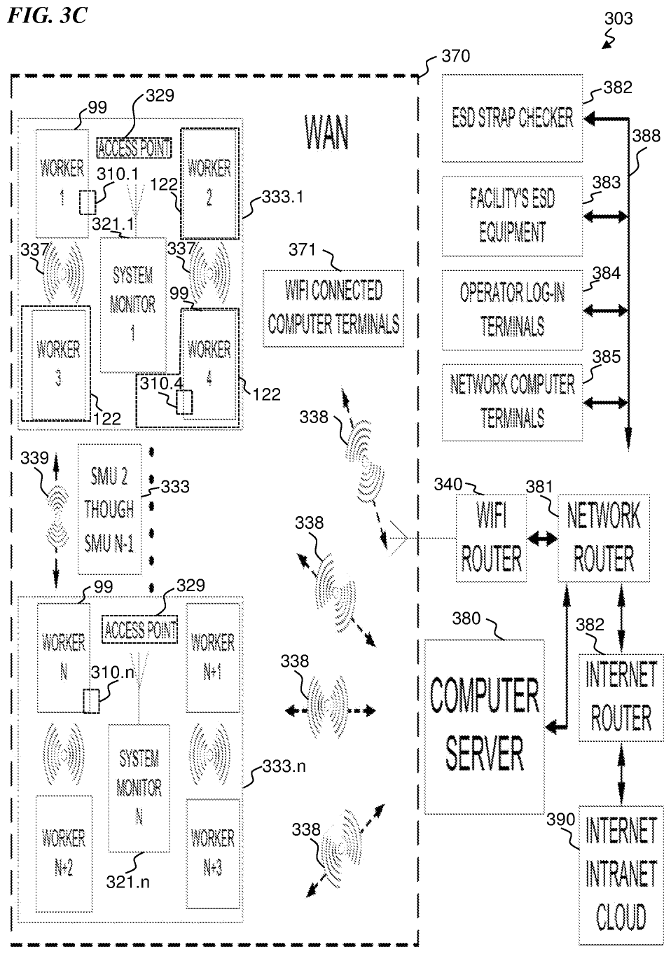

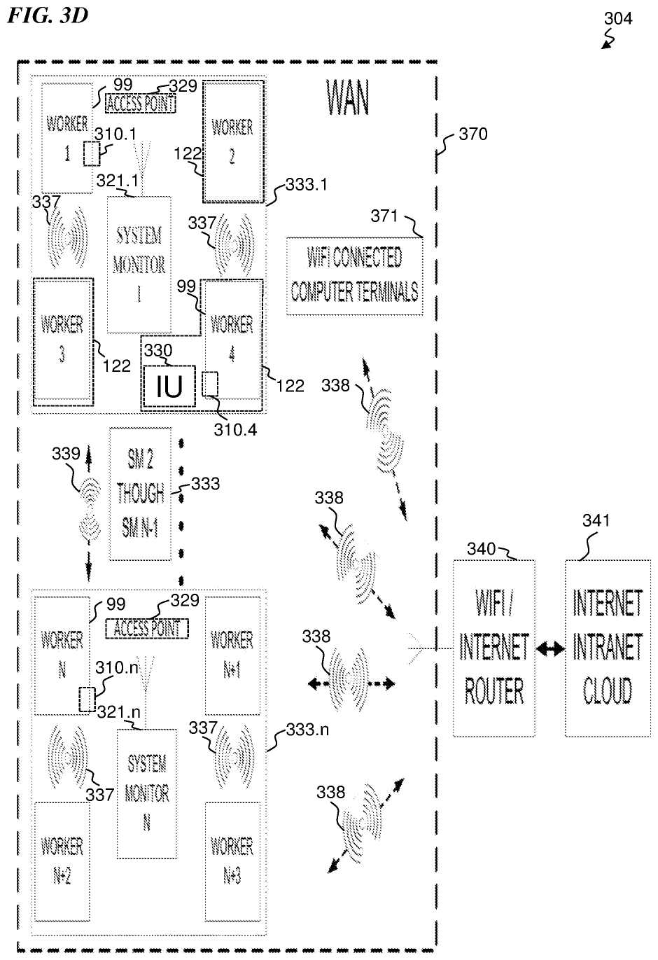

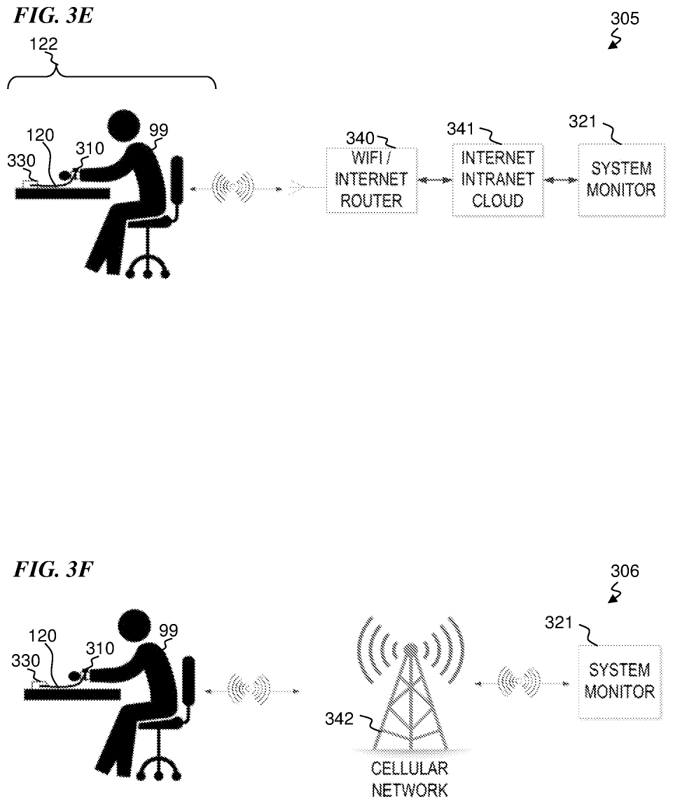

FIG. 3A is a perspective view and block diagram of a system 301 that includes a sensor unit 310 on the wrist 96 of a user 99, according to some embodiments of the present invention. In some embodiments, sensor unit 310 is implemented as a sensor unit 110 of FIG. 1A, sensor unit 170 of FIG. 1B or any other sensor unit described herein. In some embodiments, sensor unit 310 includes a display 311, and in some embodiments, display 311 includes a liquid-crystal display (LCD), optionally including touch-screen input capability. In some embodiments, when the methods and systems of the present invention are incorporated in facility-wide applications, each worker 99 is assigned and provided a sensor unit 310. Each sensor unit 310 aggregates data and transmits the data to a system monitor 321 (such as system monitor 221 of FIG. 2, but with optional additional enhancements). In some embodiments, the transmission includes data 331 transmitted directly from sensor unit 310 to system monitor 321. In other embodiments, the transmission includes data 333 communicated from sensor unit 310 to the user's smart phone 322, and then data 334 communicated from user's smart phone 322 to system monitor 321. In yet other embodiments, the transmission includes data 335 communicated from sensor unit 310 to interface unit 330 at a work area, and then data 336 communicated from interface unit 330 to system monitor 321. In some embodiments, the data (i.e., 331, 333, 334, 335 and/or 336) includes the unique ID of the sensor unit 310, the unique ID of the interface unit 330, the time of connection and/or disconnection between sensor unit 310 and interface unit 330, and/or other parameters such as a value of the measured skin resistance and the like. In some embodiments, a system management application 360 (e.g., in some embodiments, software) gathers data from one or more system monitors 321 and generates reports for the company's or customer's management and/or for compliance to a given standard (for example, ANSI/ESD S20.2). In some embodiments, system management application 360 executes in a processor inside system monitor 321; while in other embodiments, system management application 360 executes in a processor at a remote location or “in the cloud,” i.e., via computer time leased from a server “farm” connected across the internet.

FIG. 3B is a data flow and management diagram of a system 302, according to some embodiments of the present invention. In some embodiments, system 302 includes one or more system monitors 321 (e.g., 321.1 . . . 321.n), each system monitor 321 gathering data from one or more sensor units 310 (e.g., sensor units 310.1, 310.2, 310.3 each coupled to transmit its data to system monitors 321.1. and 310.x−1, 310.x, 310.x+1 each coupled to transmit its data to 321.n). In some embodiments, each sensor unit 310 is configured to gather and aggregate the unique ID and/or other parameters from its associated interface unit 330 (e.g., sensor unit 310.1 gathering and transmitting data from interface unit 330.1 to its system monitor 321.1, sensor unit 310.2 gathering and transmitting data from interface unit 330.2 to its system monitor 321.1, and sensor unit 310.x gathering and transmitting data from interface unit 330.y to its system monitor 321.n). In some embodiments, a software system-management application 360 gathers the data from one or more system monitors 321 and generates a compliance report for a selected set of users 99 (each represented by their own personal sensor unit 310) and a selected set of work areas (each represented by their own interface unit 330).

FIG. 3C is a block diagram of a networked ESD compliance system 303, according to some embodiments of the present invention. FIG. 3C illustrates an example of a facility-wide implementation using local RF (radio frequency)/wireless transceiver connections 337 between sensor units 310 (e.g., 310.1 . . . 310.4) of workers 99 at their respective workstations 122 and localized system monitors 321 (e.g., 321.1 through 321.n). In some embodiments, these system monitors 321 communicate with or via their respective local WiFi circuit 329 (in some embodiments, a WiFi range extender (communicating via a wireless connection to a centralized WiFi router 340) or an access point (connected via a wired connection (not shown) to network 388) for each work cell 333 (e.g., 333.1 . . . 333.n) to connect to the company's network (e.g., in some embodiments, a Wide Area Network (WAN)) 370. In some embodiments, other ESD equipment 383 and ESD strap checkers 382 may optionally be connected to the network by wired connections 388 (e.g., ethernet or the like) or by wireless communications to the WAN 370. The data from one or more system monitors 321 is communicated 338 to wireless router 340 (e.g., in some embodiments, by WiFi connections), consolidated in computer server 380 and managed with a software application (such as application 360 shown in FIG. 3B). In some embodiments, other terminals in the network, including WiFi-connected terminals and/or tablets 371, operator login terminals 384, and wired network terminals 385, execute application software (such as system-management application 360) to monitor, manage, and record ESD compliance data.

FIG. 3D is a block diagram of a cloud-based ESD compliance system 304, according to some embodiments of the present invention. FIG. 3D illustrates another example of collecting worker ESD data within work cells 333, in some embodiments, monitoring and aggregating work-cell (or work-area) data with a local system monitor 321 and using the wide area network (WAN) 370 to transmit data to a remote “cloud-based” hardware system 341 at a location with ESD system management software 360 (see FIG. 3B). In some embodiments, the sensor unit 310 of each worker 99 or the interface unit 330 of each work area 122 contains a WiFi transceiver and directly communicates with the local system monitor 321 through the facility's WiFi router 340. In other embodiments, the local system monitor 321 may also or alternatively communicate directly with sensor unit 310 through WiFi router 340 using an internet or intranet (“cloud-based”) connection 341.

FIG. 3E is a block diagram of a system 305 using a first connectivity method to communicate with a system monitor 321, according to some embodiments of the present invention. In some embodiments, system monitor 321 communicates with the sensor unit 310 through a network connection from sensor unit 310 to WiFi or internet router 340 connected to the internet or intranet “cloud” 341. In some such embodiments, system monitor 321 is implemented as a software application running on a cloud-based server leased as needed per a leased-computer-time contract. In some embodiments, the sensor unit 310 of each worker 99, or the interface unit 330 of each work area 122, contains a cell-phone transceiver that communicates with the facility's cellular provider's network (or, in other embodiments, sensor unit 310 of each worker 99, or the interface unit 330, communicates (e.g., using Bluetooth or WiFi) to the smartphone (not explicitly shown) of the user 99, which communicates with the facility's cellular provider's network).

FIG. 3F is a block diagram of a system 306 using a second connectivity method to communicate with a system monitor 321, according to some embodiments of the present invention. In some such embodiments, system monitor 321 communicates with the sensor unit 310 more directly through a cellular network 342.

In some embodiments, the system of the present invention includes these major elements as shown in FIG. 2 and FIG. 3A:
A sensor unit 210 or 310 worn by an individual human user 99,
A conductive strap 215 or 315,
A conducting cable 120, connecting wrist sensor unit 210 or 310 to interface unit 230 or 330, respectively,
An interface unit 230 or 330 electrically connecting via conducting cable 120 to the sensor unit 210 or 310 and electrically connecting via conducting cable 134 to an ESD or earth ground 140, and
A system monitor 221 or 321, configured to communicate with interface unit 230 or 330 and/or sensor unit 210 or 310 and to collect ESD data gathered from the interface unit 230 or 330 and/or sensor unit 210 or 310.

Description of Basic Sensor Unit 310

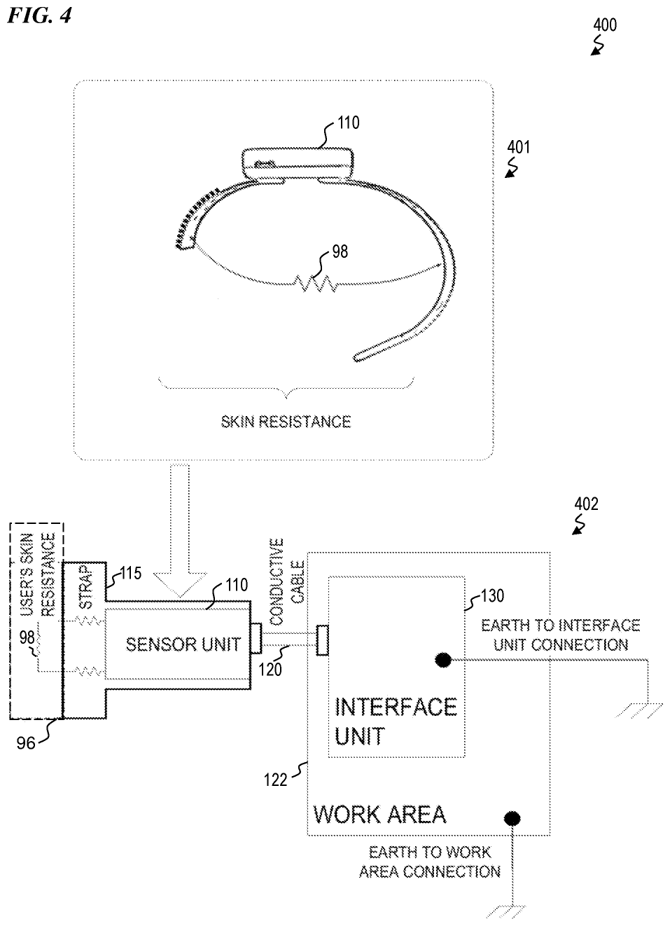

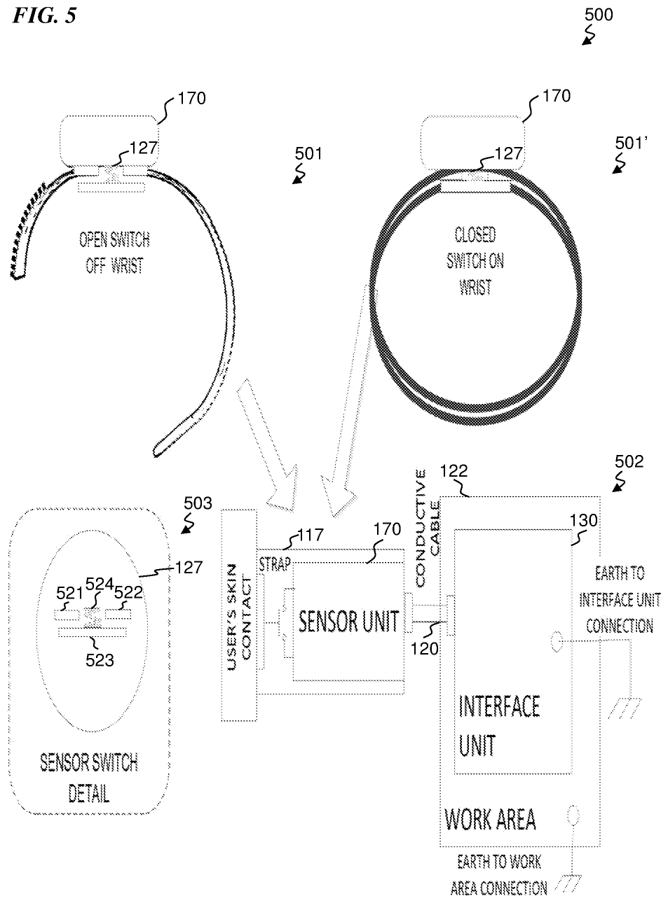

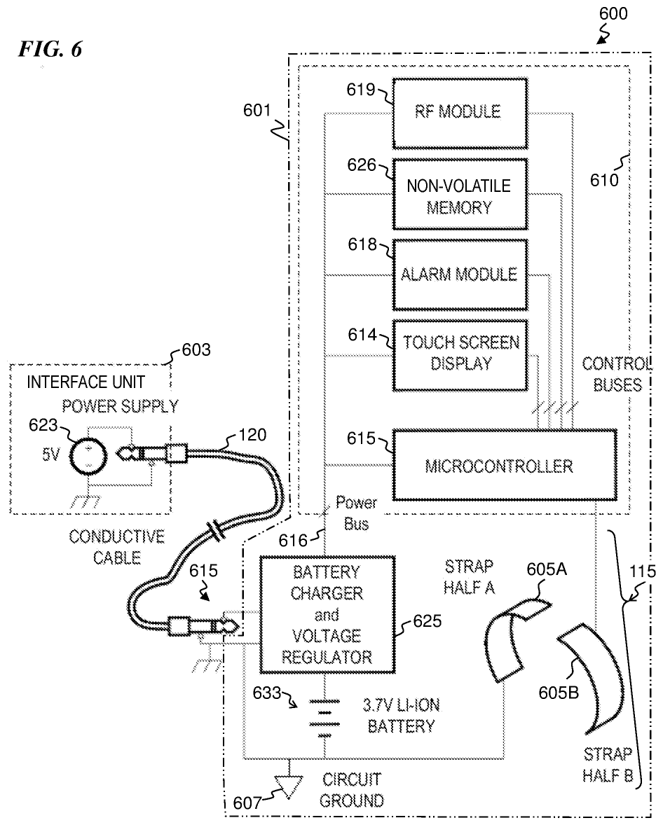

Referring again to FIG. 3A, in its most basic form, in some embodiments, sensor unit 310 includes electronic hardware and/or firmware to measure and/or validate conductive strap connection to the user's skin 96, measure or validate connection to interface unit 230, and hold a unique identifying code or ID. The sensor unit 310 is connected to, attached to, or integrated into a means or device to attach to a user's skin. A common device for skin attachment is a conductive wrist strap 315. The sensor unit 310 may use one or more of a plurality of methods to validate wrist-strap electrical connectivity to user 99, for example as shown in FIG. 4 and FIG. 5.

FIG. 4 includes a side view of conductive ESD strap assembly 401 and block diagram 402 of a system 400 that includes a sensor unit 110, according to some embodiments of the present invention. In one such embodiment, the sensor unit 110 directly measures the skin electrical resistivity 98 (see FIG. 1A), between the sensor unit 110's conductive wrist strap 115 and the user's skin 96 (as shown in FIG. 1A and FIG. 4), or conversely, the conductivity of the skin contact.

FIG. 5 includes a side view of conductive ESD strap assembly 501 (with switch 127 open), a side view of conductive ESD strap assembly 501′ (with switch 127 closed), and a block diagram 502 of a system 500 that includes a sensor unit 170, according to some embodiments of the present invention. Block diagram 503 shows a close-up view of switch 127 according to some embodiments. In the embodiment as shown in FIG. 1B and FIG. 5, momentary-contact switch 127 resides on the skin side of the sensor unit 170. When a user 99 fastens the wrist strap with appropriate tautness to electrically connect the strap to the wrist, the switch 127 closes. In some embodiments, switch 127 includes a skin-contact plate 523, a spring 524, and conductive contacts 521 and 522. Switch 127 is open when the strap is not connected to the user's wrist 96. When installing and tightening the wrist strap, force from the wrist 96 on the skin contact plate 524 compresses the spring 524. Skin contact plate 523 then electrically connects to the conductive contacts 521 and 522 of sensor unit 170. Sensor unit 170 detects the closed switch, and signals “valid-wrist-strap” connectivity to system monitor 321.

In some embodiments, sensor unit 110 and sensor unit 170 also measure and validate conductivity to an interface unit. A “safe” status (which is communicated to system monitor 321—see FIGS. 3A-3F) results when sensor unit 110 or sensor unit 170 senses that the user 99 is wearing the conductive ESD strap assembly (the combination 401 of strap 115 and sensor unit 110 or the combination 501 of strap 117 and sensor unit 170) and the conductive ESD strap is connected to an ESD-grounded interface unit 130. In various embodiments, a sensor unit optionally indicates “ESD-safe-connectivity” status with a display, indicators, and/or audible annunciators (such as a speaker or buzzer). When equipped with a wireless transmitter or transceiver, the sensor unit will communicate status to a system monitor 321.

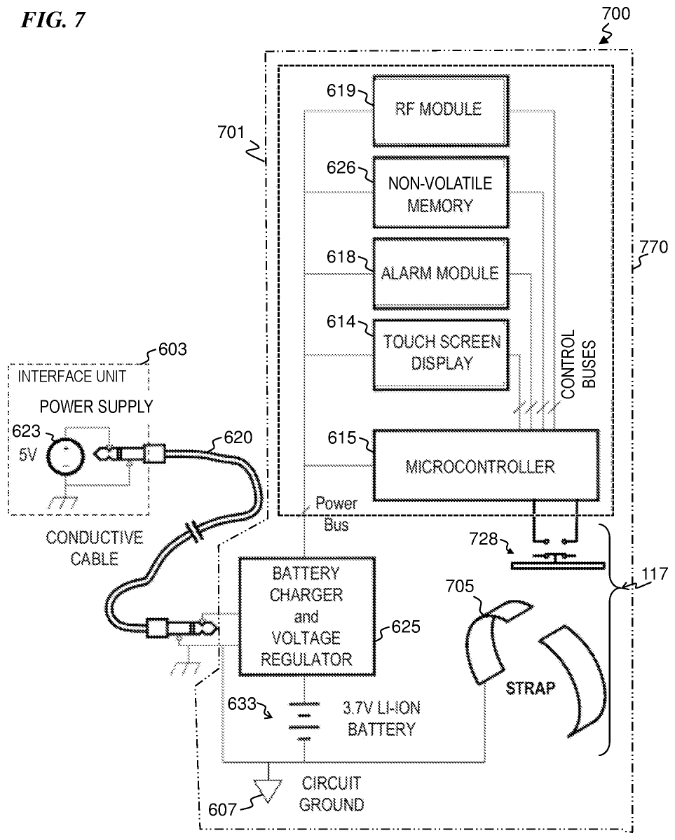

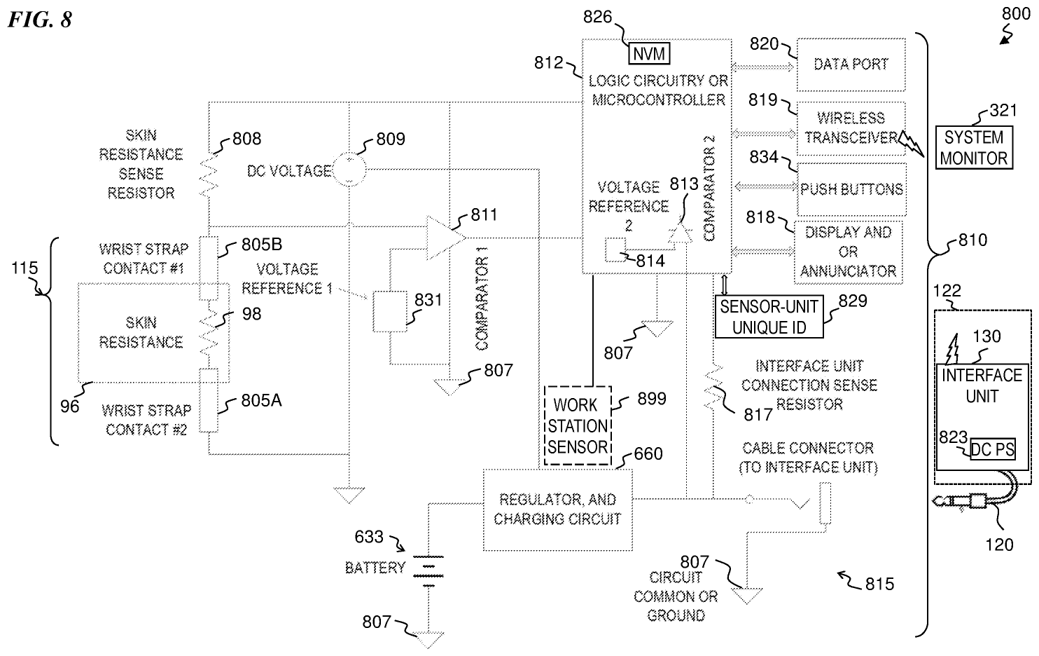

FIG. 6, FIG. 7, and FIG. 8 diagram sensor unit functional blocks.

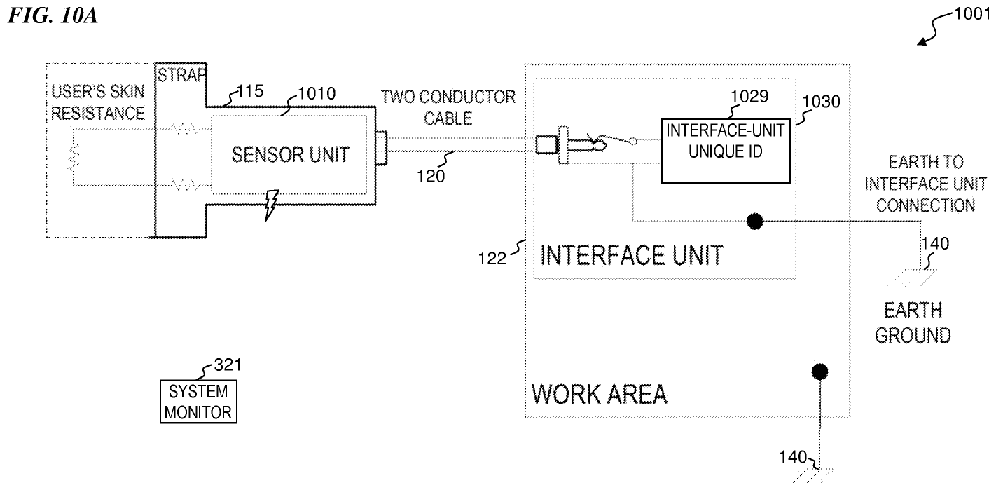

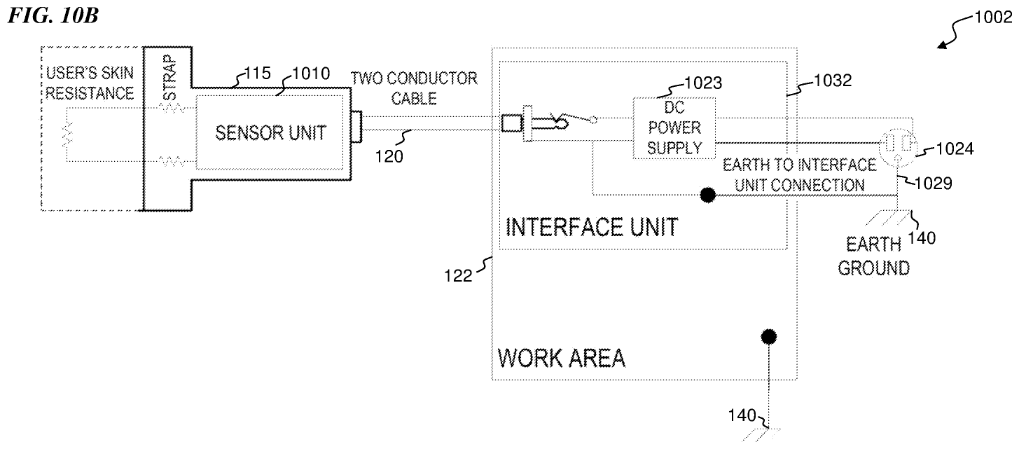

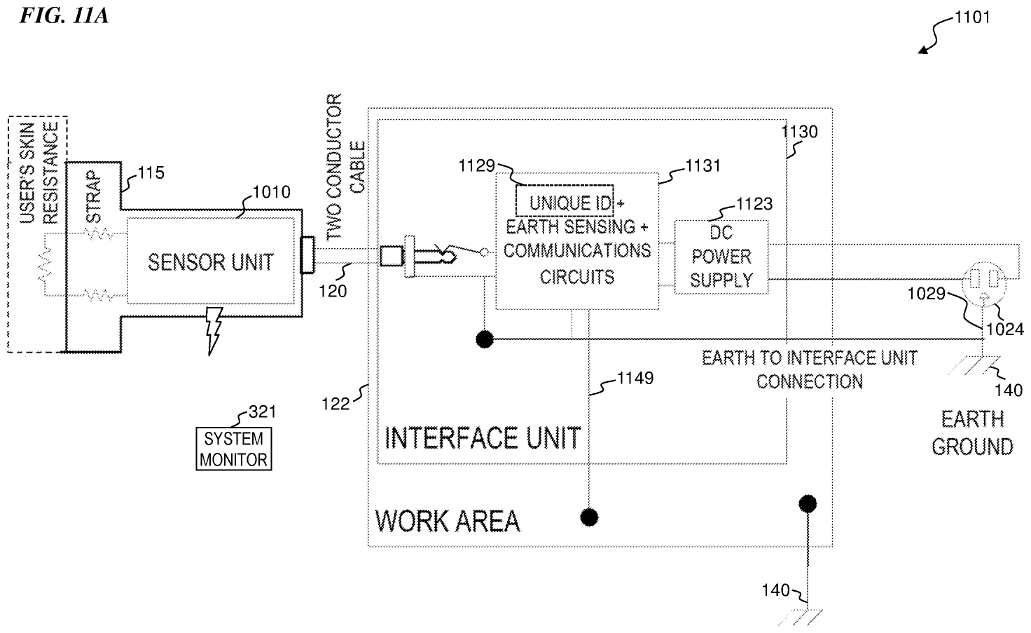

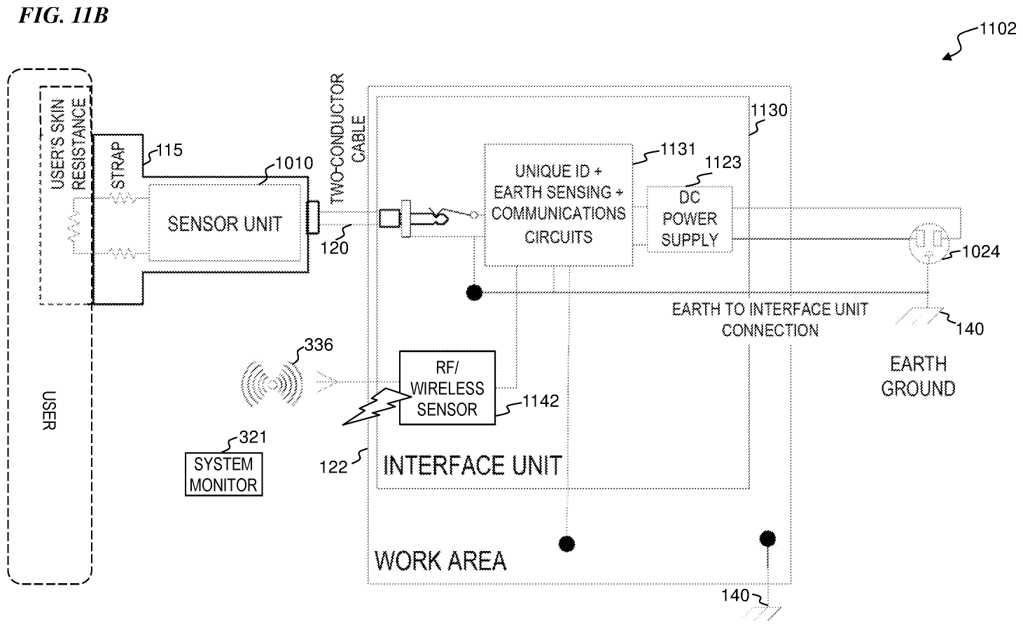

FIG. 6 is a block diagram of a conductive ESD strap system 600 that includes a skin-resistance sensor unit 610 in conductive ESD strap assembly 601, according to some embodiments of the present invention. In some embodiments, sensor unit 610 includes an RF (radio-frequency transmitter/receiver) module 619, a non-volatile memory 626 (e.g., in some embodiments, semiconductor storage), an alarm module 618, a touchscreen display 614, and a microcontroller 615. Some embodiments further include a battery charger and voltage regulator 625 and a battery 633 (such as, a lithium-ion (Li-ion) battery having a nominal voltage of 3.7V) connected between a power bus 616 and a circuit ground 607. In some embodiments, one of the strap electrical conductors, conductor 605A, of strap 115 is connected to circuit ground 607 and the other of the strap electrical conductors, conductor 605B, is electrically connected to microcontroller 615. In some embodiments, an electrically conductive cable 120 connects conductive ESD strap assembly 601 to interface unit 603, and in particular, to the power supply 623 (such as, a mains-connected power supply (such as, power supply 1023 of FIG. 10B) that provides a nominal output voltage of 5V) of interface unit 603.

FIG. 7 is a block diagram of a conductive ESD strap system 700 that includes a wrist-switch sensor unit 710 in conductive ESD strap assembly 701, according to some embodiments of the present invention. In some embodiments, conductive ESD strap assembly 701 is substantially similar to conductive ESD strap assembly 601 of FIG. 6, except that switch 728 is connected to microcontroller 615, and strap electrical conductor 705 of strap 117 is connected to circuit ground 607. When strap 117 has sufficient tautness (such as being wrapped around a body limb, such as a wrist or ankle), switch 728 changes state (closed-to-open or open-to-closed) to indicate that strap 117 is a connected to the human user 99.

In other embodiments, the function of switch 729 is replaced by an optical blood-flow or heart-rate sensor (such as commonly found in fitness-watch bracelets, e.g., FITBIT® or the like) that, when ESD strap system 700 is securely connected to the wrist of user 99, detects the varied blood-flow or heart-rate of user 99 that indicates sufficient tautness of the ESD strap system 700 to the user's wrist.

FIG. 8 is a block diagram of a conductive ESD strap sensor-unit system 800 that includes a skin-resistance sensor unit 810, according to some embodiments of the present invention. In some embodiments, conductive strap 115 fastens the sensor unit 810 to the individual's wrist 96. In some embodiments, the strap 115 includes two electrically isolated halves that are mechanically connected to hold onto a user's wrist 96. One conductive-strap half 805A is connected to the circuit common or ground 807 of sensor unit 810; this also connects the ESD or earth ground contact on the conductive cable connector 815. In some embodiments, conductive cable 120 has two conductors: one conductor connects to ESD or earth ground; the second conductor conveys power from the power supply 623 of interface unit 603 (see FIG. 6) to the battery charger and regulator circuit 825 of sensor unit 810. In some embodiments, a battery 633 within sensor unit 810 powers the electronics in the absence of supplied power from interface unit 603. In some embodiments, battery 633 allows sensor unit 810 to function when disconnected from the interface unit 603 or when connected to an interface unit without a power supply. The electronics of sensor unit 810 measure or validate skin connection by applying a voltage to skin-resistance sense resistor 808, which supplies electrical current through the second strap half 805B when both conductive strap halves contact the skin of the user, since a complete circuit forms, current flows across the skin, and measurable voltage develops across the skin-resistance sense resistor 808. In some embodiments, comparator 811 senses the resulting voltage and compares to a voltage reference 831. The voltage reference 831 establishes an acceptable skin-resistance threshold. In some embodiments, logic circuitry or firmware within a microcontroller 812 processes the state of comparator 811 for skin-resistance acceptability. In some embodiments, microcontroller 812 logs data within its non-volatile memory (NVM) 826. In some embodiments, microcontroller 812 interfaces to a display or annunciator(s) 818, or communicates the user's wrist strap ESD connectivity status via wireless transceiver 819. In some embodiments, microcontroller 812 interfaces with a wireless transceiver module 819 (e.g., in some embodiments, using infrared (IR) light or RF signals) to provide data communication with a remote system monitor 321.

In some embodiments, sensor unit 810 includes a data port 820. Among many possibilities, in some embodiments, data port 820, with a suitable connector accessed from the case of sensor unit 810, allows for direct data downloading, firmware uploading, and/or battery charging. In some embodiments, two-conductor cable 120 electrically connects the sensor unit 810 to an interface unit 130. In some embodiments, a DC power supply 823 included in, or associated with, interface unit 130 provides voltage and power to sensor unit 810. In some embodiments, electronics of sensor unit 810 senses voltage appearing at the cable connector 815 using second comparator 813 to compare to second voltage reference 814, wherein the presence of a voltage indicates connection to interface unit 130. In some embodiments, the presence of a modulated voltage indicates connection to the interface unit 130, but without an ESD or earth ground connection to the work area 122.

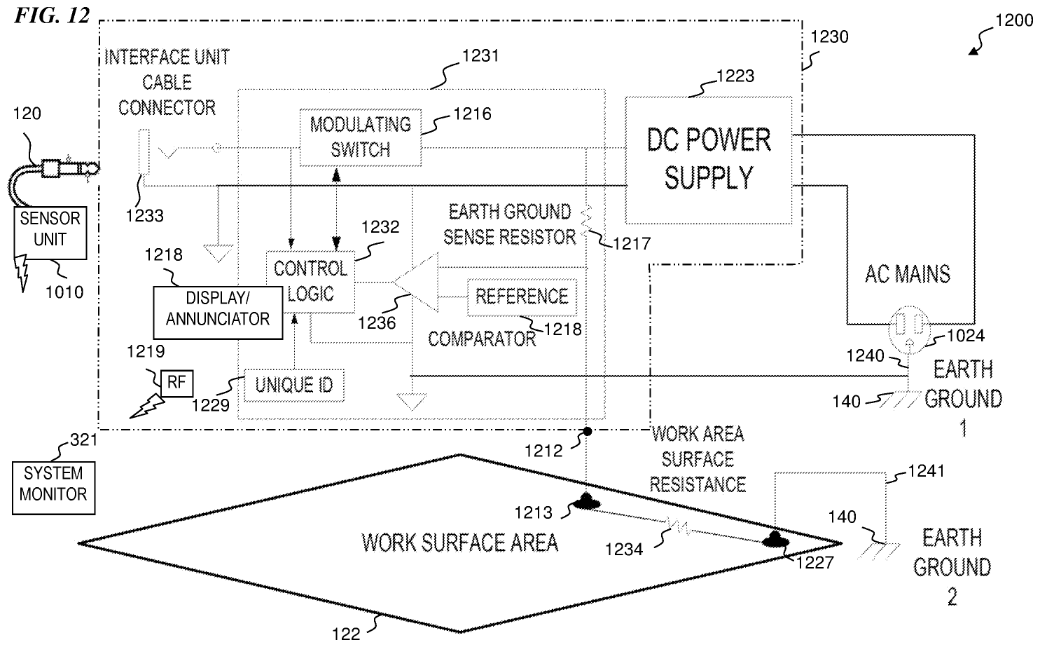

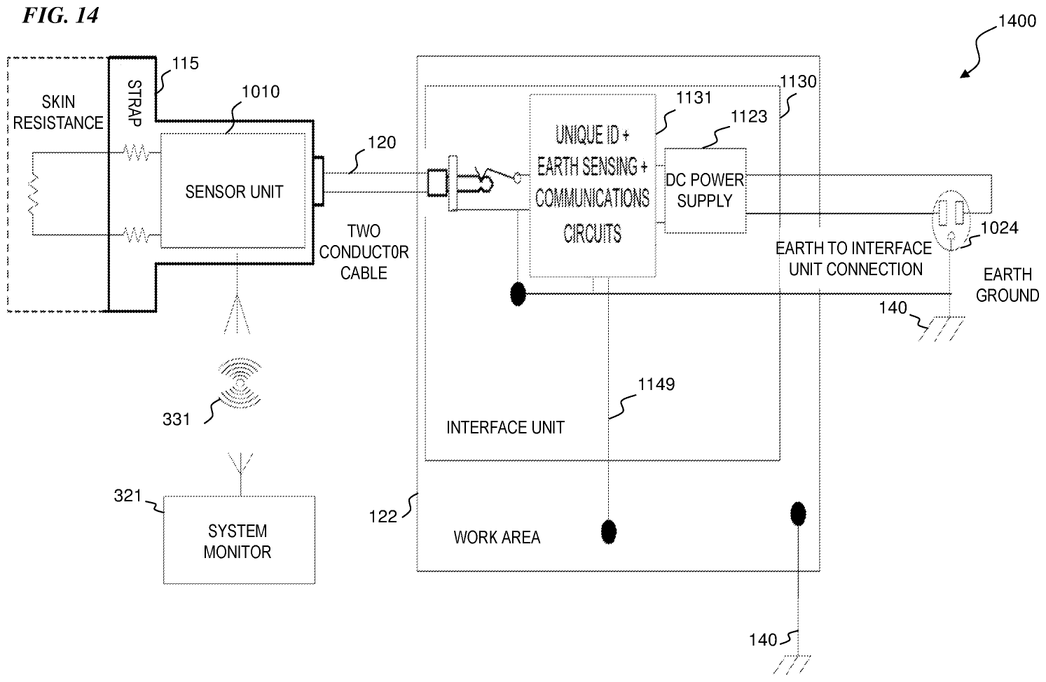

Referring to FIG. 8 and FIG. 12, in some embodiments, interface unit 130 electrically connects to an ESD or earth ground 140 directly from the three-wire outlet mains 1024 and to a contact 1227 on earth-grounded work area 122. In some embodiments, earth-sensing circuitry 1231 measures or validates an ESD- or earth-ground connection to the work area 122. In some embodiments, DC voltage from the power supply 1223 is applied to an earth-ground-sense resistor 1217. Presence of a low-resistance path on the surface of work area 122 (e.g., in some embodiments, from connection 1212 on interface unit 1230 to work-surface connector 1213 through any resistance 1234 to connection 1227 and through conductor 1241) to earth ground 140, results in a voltage drop across earth-ground-sense resistor 1217. The resulting voltage is compared (using comparator 1236) to a reference voltage 1218, which determines an acceptable threshold. There are numerous methods used by various embodiments of the present invention to communicate resulting status. In some embodiments, control-logic circuitry 1232 is used to communicate the connection status to sensor unit 1010 (sensor unit 1010 in FIG. 10A, FIG. 10B, FIG. 11A, FIG. 11B, FIG. 12, and FIG. 14 can be implemented as sensor unit 610 of FIG. 6, sensor unit 710 of FIG. 7, or sensor unit 810 of FIG. 8) from interface unit 1230. In some embodiments, control-logic circuitry 1232 processes the results and controls a modulating switch 1216.

In some embodiments, interface unit 1230 includes a display and/or annunciator 1218, and/or sensor unit 810 of FIG. 8 includes a display and/or annunciator 818, indicating to the human user or a human supervisor its status (e.g., in some embodiments, an audible beeper or voice alarm will sound if a user is at the work station 122 without connecting their sensor strap to the work station's interface unit 130 or 1230 within an allotted amount of time). In some embodiments, to communicate a “safe-connection” status indication, interface unit 130 or 1230 will maintain a constant supply voltage supply (without voltage modulation by modulating switch 1216) to the sensor unit 810. In some embodiments, to communicate an “unacceptable status,” the electronics (e.g., modulation electronics 1218) will modulate the supply voltage (e.g., in some embodiments, superimposing or multiplying an AC signal on the DC voltage). In some embodiments, “unacceptable status” includes, but is not limited to, interface unit 130 or 1230 connected to ESD ground (good connection 1240), but not connected to a Work Surface ESD ground 140 (bad connection 1241), and interface unit 130 or 1230 not connected the ESD ground 140 (bad connection 1240). In some embodiments, electronics of sensor unit 810 will detect
no voltage, indicating the sensor unit 810 is not connected to interface unit 130 or 1230;
a short circuit, indicating both conductors of the conducting cable 120 are connected to ground at the interface unit 130 or 1230;
a constant voltage, indicating a safe ESD ground connection to the interface unit 130 or 1230; and
a modulated voltage indicating unacceptable or unsafe ESD ground states.
   In some embodiments, sensor unit 810 communicates the status to the user (via display and/or annunciator 818) and system monitor 321 (via wireless transceiver 819). In some embodiments, interface unit 1230 communicates the status to the user (via display and/or annunciator 1218) and system monitor 321 (via wireless transceiver 1219).

In some embodiments, sensor unit 1010 monitors, determines, and communicates two conditions: strap-connection status, and interface-unit-connection status. Strap-connection status is either “strap connected” or “strap not connected” to the user. Interface unit (IU)-connection status is either “IU connected with unknown ESD ground status”, “IU connected with validated ESD ground status,” or “IU not connected.” An “ESD-safe status” occurs when the sensor unit strap is connected or being worn, and a validated ESD ground is sensed as connected. In some embodiments, expanded forms of the present invention include: the sensor unit 1010 logging ESD user data, displaying user ESD data or status, annunciating user ESD status, and/or communicating ESD and user data to a collection device such as system monitor 321.

In some embodiments, the sensor unit 1010 performs one or more of six functions. In some embodiments, the first function is to provide electrical connection to the individual user's skin. In one preferred embodiment, the connection is made with a conductive strap (115 or 117). In one version, the individual human user fastens the sensor unit 1010 to his or her wrist with the conductive strap (115 or 117) (see FIGS. 3A-3F). In another version, the individual fastens the sensor unit 1010 to his or her ankle with a conductive strap (115 or 117). Other embodiments directly attach the sensor unit 1010 with conductors to the user's skin at any convenient location with conductive tape or adhesive, or other skin-to-conductor fastening means. In other embodiments, sensor unit 1010 is indirectly electrically conductively attached to the user's skin through means including, but not limited to, conductive clothing or conductive shoes.

In some embodiments, the second function of sensor unit 1010 is to monitor or measure the skin connection to the sensor unit (610 or 810) and validate an electrical connection. Human skin is electrically conductive and has electrical resistance. To validate skin connection, the sensor unit (610 or 810) measures the skin resistance 98. To achieve this, some embodiments use at least two electrical contacts or connections to the skin. In some embodiments, an individual human user wears the sensor unit (610 or 810) on his or her wrist 96. In some embodiments, a strap 115 having two skin-contact electrodes establishes the skin connections. In some embodiments, the strap 115 includes two electrically isolated halves (see FIG. 6 and FIG. 8). When fastened to system unit 610 or 810, each half (605A, 605B or 805A, 805B) is electrically isolated from the other half. Alternatively, in some embodiments, an isolated conductive surface on the back side of sensor unit (610 or 810) is used in place of one of the two skin connections (605A, 605B or 805A, 805B). After fastening the strap 115 to the individual user's skin, the skin's electrical conductance bridges the two isolated conductors and forms a complete circuit. The sensor unit (610 or 810) senses or measures the skin's electrical resistance (or its electrical inverse—the skin's electrical conductance). In some embodiments, this measurement is done by inducing a current to the skin through one of the conductors 605B or 805B. The other conductor 605A or 805A connects to the common or circuit reference common or ground 607 or 807 of sensor unit 610 or 810. In some embodiments, the electronics of sensor unit 610 or 810 measures the voltage resulting from current flowing across the skin. Alternatively, in other embodiments, the electronics of sensor unit 610 or 810 applies a voltage through a strap conductor and measures the resulting current flowing across the skin. In some embodiments, the voltage or current used to measure skin resistance is an AC (alternating current) or amplitude-modulated signal (which may be superimposed on a DC voltage supply that provides power to the associated electronics), and the measurement reference voltage is also a correspondingly modulated reference signal.

In some of the following explanation, reference is made to sensor unit 810 and other items shown in FIG. 8. In some embodiments of the present invention, others of the sensor units described herein can be substituted, mutatis mutandis (with similar functions, adjusted making necessary alterations while not affecting the main point at issue).

In some embodiments, the third function of sensor unit 1010 is to include a means to connect the skin of user 99 (see FIG. 2) to an ESD ground and/or earth ground 140. In some embodiments, sensor unit 810 incorporates a connector 815 with at least one contact interfacing to a conductive cable 120. Conductive cable 120 conveys the ESD or earth-ground connection from the interface unit 1230. In some embodiments, a second conductor of conductive cable 120 is used to measure connectivity to the interface unit 1230, and optionally also used to convey power to the sensor unit 810 to power its electronics, and/or to charge and maintain a battery 633, and/or to convey data (such as connection status, the unique IDs of the sensor unit 810 or interface unit 1230, and the like).

In some embodiments, a work-station proximity sensor 899 (such as a near-field communications (NFC) or similar low-power RF signaling means) is used to sense when a sensor unit 810 and its user are within a given distance of a workstation 122, and includes circuitry and/or programming in microcontroller 812 that, once a user is detected as near a given work area but sensor unit 810 is not connected to interface unit 130 within some preset amount of time, communicates, displays, or indicates that the user 99 is present at work station 122 but is not properly connected to interface unit 930. In some embodiments, the indication is output as visible, audible, and/or mechanically oscillating indication, or vibratory and/or haptic indicators and/or annunciators. In some embodiments, the communication is done wirelessly from sensor unit 810 to a system monitor 321.

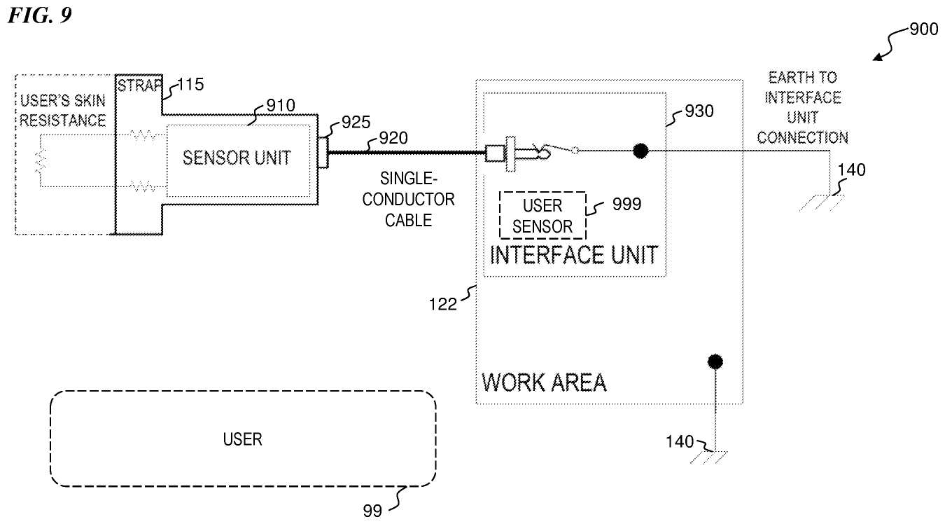

FIG. 9 is a block diagram of a system 900 that includes a skin-resistance sensor unit 910 with a single-conductor cable 920 to interface unit 930, according to some embodiments of the present invention. In some embodiments, interface unit 930 includes a sensor circuit that determines the connection of single-conductor cable 920 to interface unit 930. In some such embodiments, interface unit 930 includes a user sensor 999 (such as a passive infrared (PIR) motion sensor, or a near-field communications (NFC) or similar low-power RF signaling means) that detects the presence of a user 99 at work area 122 (i.e., within a sensing area near the work area 122), and includes circuitry and/or programming that, once a user is detected and their sensor unit 810 is not connected to interface unit 930 within some preset amount of time, communicates, displays, or indicates that the user 99 is present at work station 122 but is not properly connected to interface unit 930. In some embodiments, the indication is output as visible, audible, and/or mechanically oscillating indication, or vibratory and/or haptic indicators and/or annunciators. In some embodiments, the communication is done wirelessly to a system monitor 321.

FIG. 10A is a block diagram of a system 1001 that includes a skin-resistance sensor unit 1010 with a two-conductor cable 120 connecting to interface unit 1030, according to some embodiments of the present invention. In some such embodiments, interface unit 1030 includes a user sensor 999 (such as shown in FIG. 9) that detects the presence of a user 99 at work area 122, and includes circuitry or programming that, once a user is detected but not connected within some preset amount of time, communicates, displays, or indicates that the user is present at work station 122 but is not properly connected to interface unit 1030. In some embodiments, the indication is output as visible, audible, and/or mechanically oscillating indication, or vibratory and/or haptic indicators and/or annunciators. In some embodiments, the communication is to a system monitor 321. In some embodiments, interface unit 1030 includes an interface-unit unique ID 1029 that is communicated to sensor unit 1010. In some such embodiments, sensor unit 1010 communicates its own sensor-unit unique ID 829 (see FIG. 8) along with interface-unit unique ID 1029 to system monitor 321; in some such embodiments, a time stamp is added along with a connection/disconnection status upon each instance when cable 120 is plugged into or unplugged from interface unit 1030.

FIG. 10B is a block diagram of a system 1002 that includes a skin-resistance sensor unit 1010 with a two-conductor cable 120 connecting to interface unit 1032, according to some embodiments of the present invention. In some embodiments, interface unit 1032 includes its own power supply 1023 that obtains power (typically, relatively high-voltage AC power) from grounded mains connector 1024, and which supplies power (typically, relatively low-voltage DC power) to sensor unit 1010.

In some embodiments, the fourth function of sensor unit 1010 is to measure or validate the electrical connection of sensor unit 810 to an ESD or earth ground 140. In some embodiments, in its most-basic form, sensor unit 810 validates the presence of a cable 120 (see FIG. 9 and its description). In a system 900 using a single-conductor cable 920, sensor unit 910 verifies the presence of the cable 920 in its connector 925. This basic version assumes the user 99 has connected to a valid ESD or earth ground 140.

The next level of functionality requires a two-conductor cable (see FIG. 8 and FIG. 10A and their descriptions). With a two-conductor cable 120, sensor unit 1010 validates the integrity of connection of the two-conductor cable 120 to interface unit 1030. In some embodiments, interface unit 1030 establishes (e.g., determines) the status of the connection to an ESD or earth ground 140 or connection point. In some embodiments, sensor unit 1010 validates connectivity to interface unit 1030 by measuring the conductivity or resistance of the connection; in some embodiments, this is done by applying a DC (direct-current) voltage or a current to the non-ground contact of sensor unit connector 815 and measuring the resulting voltage with the electronics (sensor resistor 817 and microcontroller 812) of sensor unit 810. While this validates connectivity to the interface unit 130, it does not assure connection to an ESD or earth ground 140.

In another higher-functional-level form, the interface unit 1032 provides DC voltage conveyed by the two-conductor cable to the sensor unit 1010 (see FIG. 10B and its description). In some embodiments, power supply 1023 used in this version uses the earth grounding wire 1029 from a three-wire VAC (voltage with alternating current) power mains to provide an earth ground. The electronics of sensor unit 1010 senses or measures the DC voltage on the two-conductor cable. Presence of the DC voltage validates a connection to the interface unit 1032 and with a hard wired ESD or earth ground 140. In one preferred embodiment, the interface unit 1032 provides DC voltage and power to sensor unit 1010 and connects the three-wire VAC power mains earth ground 140 to the circuit common of interface unit 1032.

FIG. 11A is a block diagram of a system 1101 that includes a skin-resistance sensor unit 1010, according to some embodiments of the present invention. In some embodiments, interface unit 1130 includes DC power supply 1123, and circuit 1131 that includes interface-unit unique ID 1129 and earth-sensing and communications circuitry. In some embodiments, interface unit 1130 communicates interface-unit unique ID 1129 to sensor unit 1010. In some such embodiments, sensor unit 1010 communicates its own sensor-unit unique ID 829 (see FIG. 8) along with interface-unit unique ID 1129 to system monitor 321; in some such embodiments, a time stamp is added along with a connection/disconnection status upon each instance when cable 120 is plugged into or unplugged from interface unit 1130.

FIG. 11B is a block diagram of a system 1102 that includes a skin-resistance sensor unit 1010 with a two-conductor cable 120 to interface unit 1130, according to some embodiments of the present invention. In some embodiments, interface unit 1130 includes its own power supply 1123 that obtains power (typically, relatively high-voltage AC power) from grounded mains connector 1024, and power supply 1123 supplies power (typically, relatively low-voltage DC power) to sensor unit 1010. In some embodiments, interface unit 1130 includes its own RF/wireless module 1142, which communicates (e.g., data communication 336) between system monitor 321 and interface unit 1130.

The surface of work area 122 usually requires ESD grounding. In some embodiments, this is done with conductive tables or benches, or commercially available ESD conductive mats. The work surfaces usually have means to connect them to an earth ground 140. When interface unit 1130 also electrically connects its connection to ground 140 to a grounded ESD work surface 122, this forms a redundant connection. In one preferred embodiment, the interface unit 1130 measures or validates the work surface ESD or earth ground connection (see FIG. 11A and FIG. 11B). In some embodiments, earth-sensing circuit 1131 of interface unit 1130 measures or validates the presence of an ESD ground connection within interface unit 1130 and to the surface of work area 122. In some embodiments, interface unit 1130 signals the status of the ESD or earth ground connection of interface unit 1130 and of work surface 122 to sensor unit 1010. In some embodiments, a static DC voltage indicates a safe ESD grounding status, while a modulated DC voltage indicates a potentially unsafe ESD or earth ground status. In some embodiments, sensor unit 1010 detects the modulated supplied voltage, indicating a potentially ungrounded work surface area. This is just one method to perform this function. In other embodiments, other methods include, but are not limited to, using a third or additional conductors for communicating status, as well as other data.

In some embodiments, the fifth function of sensor unit 1010 is to communicate or display the status of the ESD connection to the individual user 99 wearing the sensor unit 810 (see, e.g., display/annunciator 818 of FIG. 8 and/or display/annunciator 1218 of FIG. 12) and/or the electrical connections of the sensor unit 810 to the grounding or ESD connection point 140. In some embodiments, minimally, the sensor unit 810 will indicate an unsafe ESD status when it senses or measures or determines a strap disconnection or a cable disconnection. In one method, the sensor unit 810 determines strap disconnection by measuring skin resistance. Elevated or too high resistance creates ESD damage risk. In some embodiments, high resistance occurs when the strap is not fastened, not fastened correctly, or when the user's skin is not effectively connected to the straps or skin contacts. In some embodiments, sensor unit 810 also indicates when it senses or measures an acceptably low resistance.

In other embodiments (see FIG. 7), sensor unit 710 determines strap disconnection by sensing the status change of the skin-side switch 728 of sensor unit 710. In one example embodiment, switch 728 is closed when the strap is properly secured to the wrist, but when the strap is loosened, removed, or disconnected, switch 728 opens. In other embodiments, switch 728 is opened when the strap is properly secured to the wrist, but when the strap is loosened, removed, or disconnected, switch 728 closes. In some embodiments, sensor unit 710 detects the state change of switch 728 and indicates the ESD safety status. Similarly, the sensor unit 710 or 810 also indicates status of its connection to conductive cable 120, the connection to the interface unit 1230, connection to an ESD or earth ground 140 at the interface unit 1230 and/or the work surface 122. This indication provides the user assurance of ESD protection. The communication, display, or indication may include, but is not limited to a visible, audible, and/or mechanically oscillating or vibratory and/or haptic indicators and/or annunciators. In a preferred embodiment, the sensor unit 810 incorporates an LCD display 829 to visually show ESD status, as well as other value-added information including, but not limited to, time, work-related information, and connection status to system monitor 321.

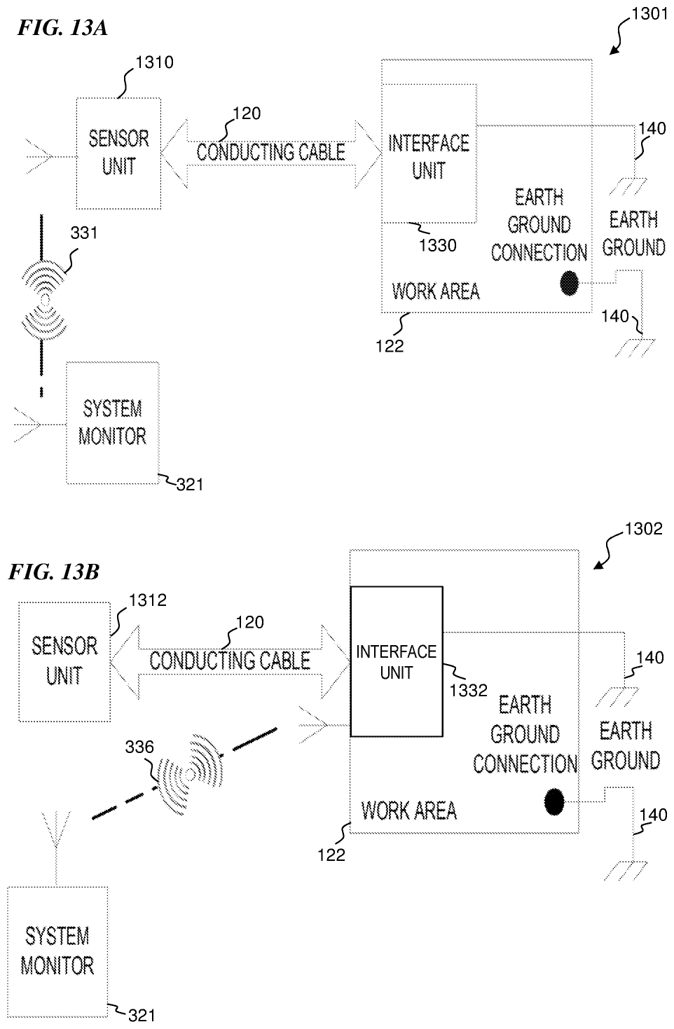

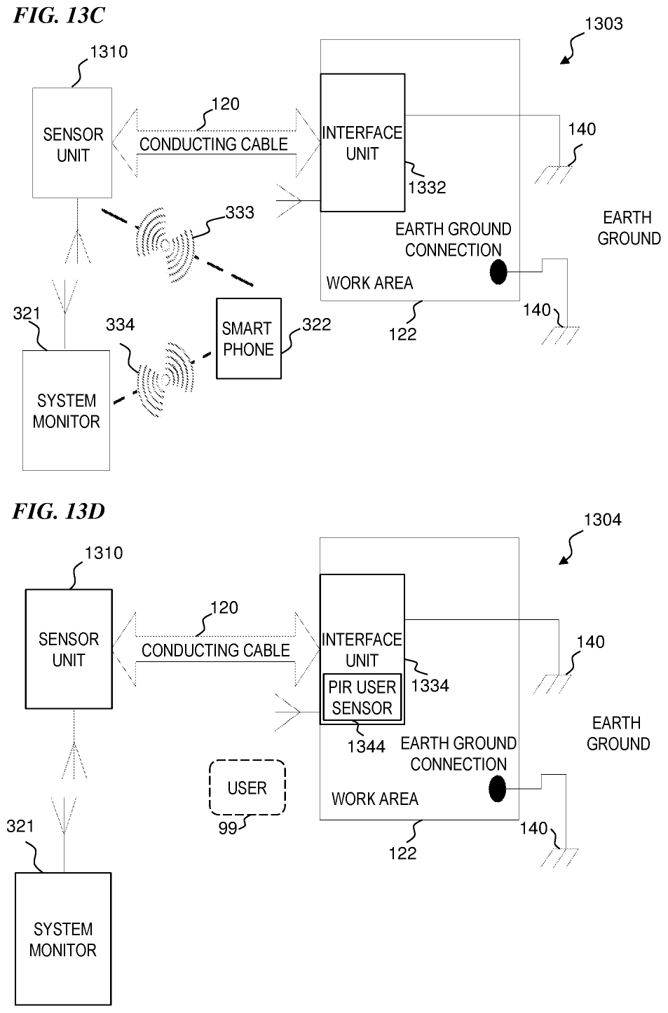

In some embodiments, the sixth function of sensor unit 1010 is to collect the wearer's ESD status data and communicate ESD connectivity data to a system monitor 321 (see FIG. 13A, FIG. 13B, FIGS. 13C and 13D). In some embodiments, system monitoring 321 collects the ESD data and communicates with the sensor unit 810 and other system components. In some embodiments, the communication method includes wired or wireless methods. In some embodiments, data transmissions of sensor unit 810 occur continuously, at intervals, or as a batch upload to a system monitor 321. In some embodiments, the communication occurs directly to system monitor 321 through a wireless signal 331 (see FIG. 13A) or through a wired data cable (not shown). In some embodiments, sensor unit 810 also indirectly communicates user-connectivity data through the interface unit 1332 (see FIG. 13B)—in this version, conducting cable 120 transmits and receives ESD connectivity data to the interface unit 1332. In some embodiments, interface unit 1332 then communicates with a wired or wireless connection 336 to system monitor 321.

Interface Unit Description

In its basic form, in some embodiments, the Interface Unit performs two principal functions (see interface units 930, 1030, and 1032 of FIG. 9, FIG. 10A, and FIG. 10B, respectively). The first function is to provide an electrical connection to an ESD or earth ground 140. The interface unit 930, 1030, or 1032 connects directly to an earth ground or to an ESD ground 140. The second function is to provide electrical connection to the conducting cable 920 or 120 that connects the sensor unit 910 or 1010 to the interface unit 930, 1030, or 1032. In embodiments in which two-conductor conducting cable 120 connects the sensor unit 1010 to the interface unit 1030, one conductor of conducting cable 120 connects the sensor unit 1010 to the ESD Ground contact 140 on the interface unit 1030. The other conductor of conducting cable 120 provides power and conveys data between the sensor unit 1010 and the interface unit 1030. In some embodiments, interface unit 1030 contains memory or a method to hold an interface unit unique identifier (ID) 1029. In some embodiments, when interface unit 1030 does not have power, the sensor unit 1010 can provide power and poll the IDs from a memory device using the two-conductor cable 120.

In some embodiments, a third function of the interface unit (e.g., interface unit 1032) is to provide power for the sensor unit 1010 (see FIG. 10B). Typically, but not necessarily, the interface unit 1032 resides on or is attached to a conductive work surface 122. In some embodiments, work surface 122 includes a conductive bench top or conductive mat on a bench top, a conductive floor, a cabinet and/or equipment with conductive surfaces or contact points.

In these applications and in some embodiments, an additional fourth function of the interface unit measures or validates an ESD or earth grounded work surface and communicates status (see FIG. 11A). In this embodiment, interface unit 1130 contains an AC (alternating current) to DC (direct current) power supply 1123. In some embodiments, a three-wire AC-voltage power connector 1024 connects to the power supply 1123 of interface unit 1130. The third-wire earth ground conductor 1029 connects interface unit 1130 to earth ground 140 through the AC power mains connector 1024 being properly plugged into a corresponding socket at work area 122. In some embodiments, interface unit 1130 has circuitry 1131 to sense and verify an ESD ground connection 1149 to the work area 122. In some embodiments, work surface 122 is grounded with the ESD ground of interface unit 1130.

In some embodiments, an optional fifth function of the interface unit is to collect and transfer sensor unit data to system monitor 321. This may be done with a wired or wireless connection from the interface unit 1130 to system monitor 321 (see FIG. 2, FIG. 11B, and FIG. 13B). In one preferred embodiment, the interface unit 1130 houses an AC (alternating current) to DC (direct current) power supply 1123. In some embodiments, a three-wire VAC power connector 1024 connects to the interface unit's power supply 1123. The third wire earth ground conductor 1029 connects the interface unit 1130 to earth ground 140 through the AC power-mains connector 1024.

Conducting Cable Description

In some embodiments, the conducting cable 120 or 920 provides the means to electrically connect the sensor unit to the interface unit. In its most basic form, a single-conductor cable 920 connects the sensor unit 910 to earth or ESD ground 140 through an interface unit 930 (see FIG. 9). Versions with expanded capability and functionality use a plurality of conductors. In one preferred embodiment, the conducting cable 120 has two conductors. One conductor of conducting cable 120 provides voltage, sources current to the sensor unit 1010, and conveys data between the interface unit 1030 and the sensor unit 1010. The second conductor of conducting cable 120 returns current and establishes a connection to an ESD or earth ground 140. In other embodiments, additional conductors are provided for additional control, redundancy, and/or data communication.

System Monitor 321

In some embodiments, system monitor 321 performs two basic functions. The first function is to collect the sensor-unit user's ESD connectivity and safety data. The second function is to process and report data, and report the user's ESD issues and compliance to a supervisor, manager, or ESD-compliance manager. In some embodiments, system monitor 321 communicates with and collects data received from one or multiple sensor units and/or interface units. In some embodiments, application software 360 (see FIG. 3B) within system monitor 321 processes the collected status data, and monitors and records users' ESD compliance.

In some embodiments, a manager or supervisor will provide a user 99 with a sensor unit 810 with a unique sensor-unit ID 829 (see FIG. 8). In some embodiments, using the software 360, the manager or supervisor person will assign or associate the unique sensor-unit ID 829 to a specific user 99. In some embodiments, sensor unit 810 communicates (e.g., status data) to system monitor 321 every time there is a state change. The states and/or data optionally include, but are not limited to, “sensor unit power is on,” “sensor unit power is off,” “sensor unit strap is not attached,” “sensor unit strap is attached,” “sensor unit is connected to a known ESD grounded interface unit,” “sensor unit is connected to an interface unit with an indeterminate ground connection,” and/or “sensor unit battery level.” In some embodiments, application software 360 keeps track of time and records state changes for each user 99 (e.g., to which work area 122 was their sensor unit 810 connected to or disconnected from and when, and was the ESD connection sufficient to protect the products which the user was touching). In some embodiments, application software 360 communicates status and alerts to human supervisors, managers, or the ESD-compliance manager.

In some embodiments, system monitor 321 has several possible hardware platform options including but not limited to computers, smart phones, tablets, smart watches, internet cloud-based servers, or any equipment with a means to receive or transfer data and display results. The means to collect data may be through a wired or wireless connection between sensor unit 810 or interface unit 1230 or other ESD safety equipment, including commercially available equipment, and system monitor 321. In some embodiments, the hardware platform requires software and firmware to create reports, alerts, and ESD-management tools. In embodiments where sensor units have displays and wireless transceivers, a third function allows system monitor 321 to communicate with and control sensor units 810. In one embodiment, system monitor 321 is implemented in a computer terminal that has a wireless radio-frequency (RF) transceiver. The RF receiver logs multiple user ESD data from respective computer terminals. In some embodiments, application software 360 displays user data. The software 360 generates alerts when ESD disconnection occurs. In some embodiments, the present invention uses one of many possibilities for alerting. In some embodiments, alerts are in the form of an email, SMS text message, or an automated phone call. From the computer terminal having system monitor 321 functionalities, a human manager may remotely control or configure the computer terminal and/or the interface units 1230 with which system monitor 321 is connected. A human manager can also communicate directions to a user 99 of a particular sensor unit 810. In another embodiment, system monitor 321 is implemented as an internet-based cloud software application accessible and controllable from a cell phone, tablet, or remote computer terminal.

Functional Description of Some Preferred Embodiments

FIG. 13A is a block diagram of a system 1301 that uses communications to a system monitor 321 via a sensor unit 1310, according to some embodiments of the present invention. In some embodiments, sensor unit 1310 communicates directly to system monitor 321 through a wireless signal 331 or through a wired data cable (not shown). In some such embodiments, the serial number (ID) of interface unit 1330 is transmitted to sensor unit 1310 via conducting cable 120, which then communicates the ID of interface unit 1330 and ID of sensor unit 1310 to system monitor 321. In some embodiments, the communicated ID data, along with connect events and disconnect events, and optionally parameters such as measured skin resistance and measured interface unit resistance to the earth ground 140, are recorded in non-volatile memory along with timestamps for each event and piece of information.

FIG. 13B is a block diagram of a system 1302 that uses communications to a system monitor 321 via an interface unit 1332, according to some embodiments of the present invention. In some embodiments, interface unit 1332 communicates directly to system monitor 321 through a wireless signal 336 or through a wired data cable (not shown). In some such embodiments, the serial number (ID) of sensor unit 1312 is transmitted to interface unit 1332 via conducting cable 120, which then communicates the ID of interface unit 1332 and the ID of sensor unit 1312 to system monitor 321. In some embodiments, the communicated ID data, along with connect events and disconnect events, and optionally parameters such as measured skin resistance and measured interface unit resistance to the earth ground 140, are recorded in non-volatile memory along with timestamps for each event and piece of information.

FIG. 13C is a block diagram of a system 1303 that uses communications to a system monitor 321 via a smartphone 1360 and a sensor unit 1313, according to some embodiments of the present invention. In some embodiments, sensor unit 1310 communicates 333 to a user's smart phone 322, which then wirelessly communicates 334 to system monitor 321. In some embodiments, interface unit 1332 communicates to the user's smart phone 322, which then wirelessly communicates 334 to system monitor 321.

FIG. 13D is a block diagram of a system 1304 that uses a proximity detector 1344 (such as a PIR (passive infrared) motion sensor as are well-known in the art, or the like) that detects whether there is a user at the workstation but not connected to the interface unit, according to some embodiments of the present invention. In some embodiments, user-proximity detector 1344 provides information to a microprocessor in interface unit 1334 that detects disconnected-user events associated with a detected presence of a user 99 at his or her respective work station 122 without also detection of the connection of cable 120 to sensor unit 1310. If the user has not connected his or her wearable ESD device 1310 to the respective ESD interface device within a preset amount of time, and a disconnected-user event is communicated to ESD system monitor 321. In some embodiments, ESD system monitor 321 is programmed to record disconnected-user events and to record associated timestamps for all disconnected-user events. In some embodiments, ESD interface unit 1344 also includes a visual and/or audio alarm output that is activated to indicate a disconnected user present after the preset amount of time.

FIG. 14 is a block diagram of a system 1400 that uses communications to a system monitor 321 via a sensor unit 1010, according to some embodiments of the present invention. In some embodiments, system 1400 monitors and validates conductivity between users' skin to an ESD or earth ground 140 and users' work areas 122. A user 99 wears the sensor unit 1410 (which in some embodiments is as shown as sensor unit 810 in FIG. 8) on his or her wrist. In some embodiments, sensor unit 810 has a display or indicators 818 showing connectivity status of, but not limited to, wrist strap connection to user's skin, connection of cable 120 to the sensor unit 810, connection to the interface unit 1230, and connection of ESD or earth ground 140 to the work area 122. In some embodiments, sensor unit 810 also includes audible, tactile, and/or haptic user feedback to the user 99 to alert for an unsafe ESD condition. In some embodiments, interface unit 1130 provides connection to a safe ESD or earth ground 140, provides power to the sensor unit 810, measures or validates connection between ESD or earth ground 140 and work area 122, and communicates status to the sensor unit 810. In some embodiments, sensor unit 810 incorporates a wireless RF transceiver to wirelessly connect 331 data transmission to a system monitor 321 which is also equipped with a wireless RF transceiver. In some embodiments, sensor unit 810 logs a user's ESD status data and transmits it to system monitor 321. In some embodiments, system monitor 321 collects data from a plurality of users, each wearing their own respective sensor unit 810.

In this embodiment, an individual wears the sensor unit 401 or 501 on his or her wrist (see FIG. 4 and FIG. 5). A conductive strap fastens the sensor unit to the individual's wrist. FIGS. 10A, 10B, 11A and 11B depict block diagrams with two of many different methods of the present invention to validate whether users are wearing or are connected to their ESD wrist straps of the present invention.

FIG. 8, again, is a block diagram of a sensor unit 810 that provides detail to the skin-resistance measurement method of some embodiments of the present invention. A conductive strap fastens sensor unit 810 to the individual's wrist. In some embodiments, the strap includes two electrically isolated halves that are mechanically connected to hold onto a user's wrist. One conductive strap half 805A is connected to the sensor unit's circuit common or ground 807. This also connects the ESD or earth ground contact on the conductive cable connector 815. In some embodiments, the conductive cable 120 has two conductors. One conductor conveys ESD or earth ground, and the other, second conductor conveys power from the power supply 1223 of interface unit 1230 (see FIG. 12) to the battery charger and regulator circuits 825 of sensor unit 810. In some embodiments, a battery 833 within sensor unit 810 powers the electronics in the absence of power supplied by interface unit 1230. In some embodiments, battery 833 allows sensor unit 810 to function when disconnected from interface unit 1230 or when connected to an interface unit without a power supply.

In some embodiments, the electronics of sensor unit 810 measure or validate skin connection by applying a voltage to the second strap half 805B. When both conductive strap halves contact the skin, a complete circuit forms, current flows across the skin, and measurable voltage develops across the skin-resistance-sense resistor 808. A comparator 811 senses the resulting voltage and compares to a voltage reference 831. The voltage reference 831 establishes an acceptable skin resistance threshold. In some embodiments, logic circuitry or firmware within a microcontroller 812 processes the state of comparator 811 for skin-resistance acceptability. In some embodiments, microcontroller 812 logs data within its non-volatile memory 826. In some embodiments, microcontroller 812 interfaces to a display and/or annunciator(s) 818 and/or communicates (e.g., via wireless transceiver 819) the user's wrist strap ESD connectivity status to system monitor 321. In some embodiments, the sensor unit 810 includes a data port 820. Among many possibilities used by various embodiments of the present invention, a data port 820 with a connector accessed from the case, allows for direct data downloading (into system monitor 321 from sensor unit 810), firmware uploading (of software updates into microcontroller 812), and battery charging.

In some embodiments (see FIG. 8 and FIG. 12), two-conductor cable 120 electrically connects sensor unit 810 to an interface unit 1230. In some embodiments, a DC power supply 1223 included or associated with interface unit 1230 provides voltage and power to sensor unit 810. In some embodiments, electronics in sensor unit 810 senses voltage appearing at the cable connector 815 using comparator 813 to compare to voltage reference 814. The presence of voltage indicates connection to the interface unit 1230. The presence of a modulated voltage indicates connection to interface unit 1230, but with an ESD or earth ground or disconnection to the work area 122. In some embodiments, interface unit 1230 electrically connects to an ESD or earth ground 140 directly from the three-wire outlet mains 1024 and to a contact 1227 on earth-grounded work area 122.

In some embodiments, earth-sensing circuitry 1231 measures or validates an ESD or earth ground connection to the work area 122. DC voltage from the supply 1223 is applied to an earth ground sense resistor 1217. Presence of a low resistance to earth ground on the surface of work area 122 results in a voltage drop across the earth ground sense resistor 1217. The resulting voltage is compared to a reference voltage 1218, determining whether the connection meets an acceptable threshold value for grounding.

The present invention uses one or more of numerous methods to communicate the resulting status. The embodiment of FIG. 12 uses control logic circuitry 1232 to communicate the connection status to the sensor unit 810. In some embodiments, control logic 1232 processes the results and controls a modulating switch 1216.

In some embodiments, interface unit 1230 includes a display 1218 indicating its status. To communicate a safe connection status, interface unit 1230 maintains a constant supply voltage supply to the sensor unit 810. For an unacceptable status, the Interface Unit's electronics will modulate the supply voltage. Unacceptable status includes but is not limited to “interface unit connected, but not connected to a work surface ESD ground,” and “interface unit not connected the ESD ground.” In some embodiments, electronics of sensor unit 810 detect:
no voltage, indicating the “sensor unit is not connected to interface unit”;
a short circuit, indicating “both conductors of the conducting cable are connected to ground at the interface unit”;
a constant voltage, indicating “a safe ESD ground connection to the interface unit”; and/or
a modulated voltage, indicating “an unacceptable or unsafe ESD ground state”.
   In some embodiments, sensor unit 810 communicates the status to user 99 and system monitor 321.

Sensor-Unit Schematic Description

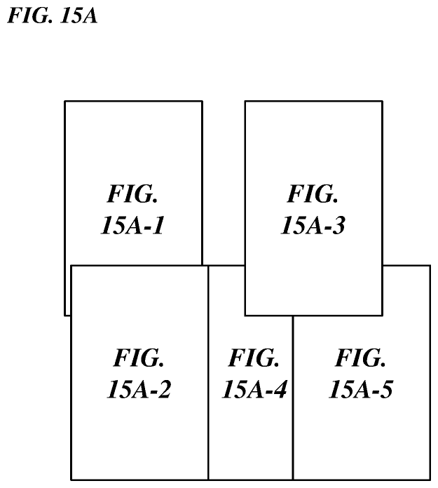

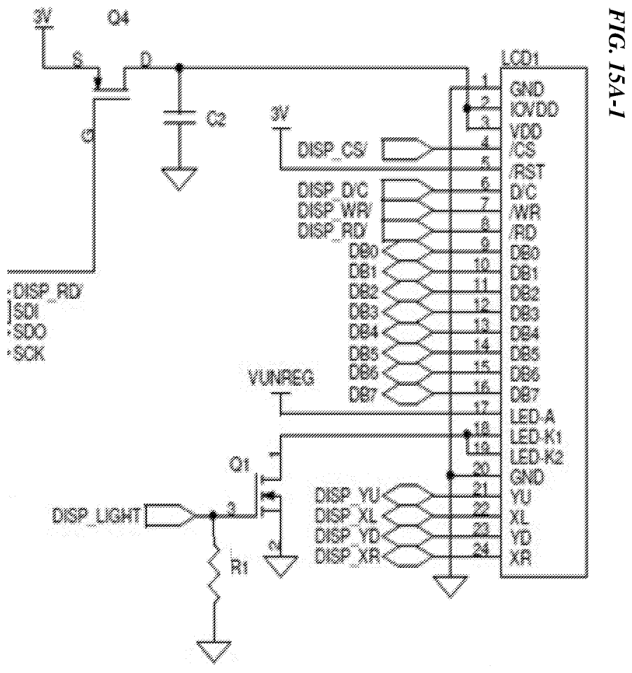

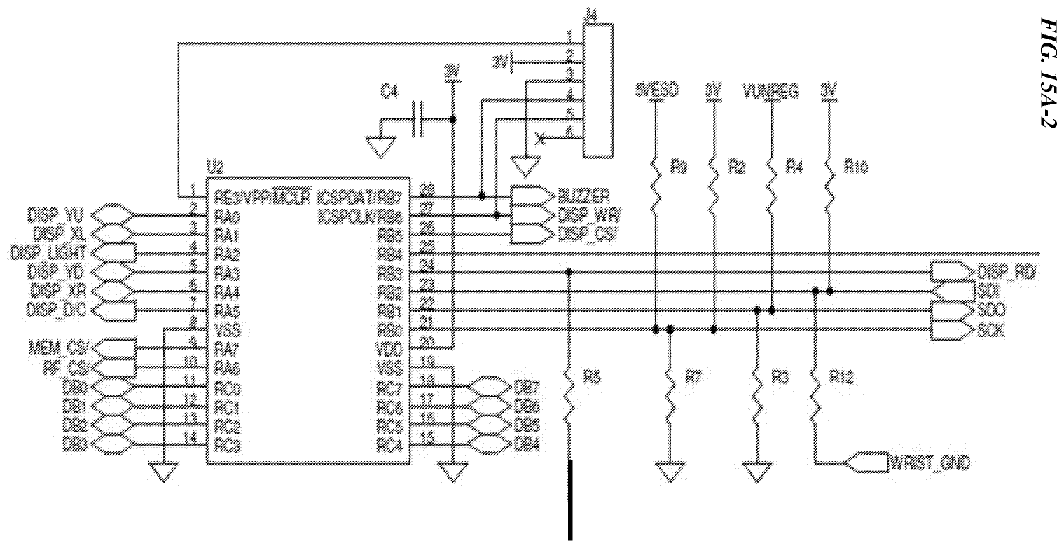

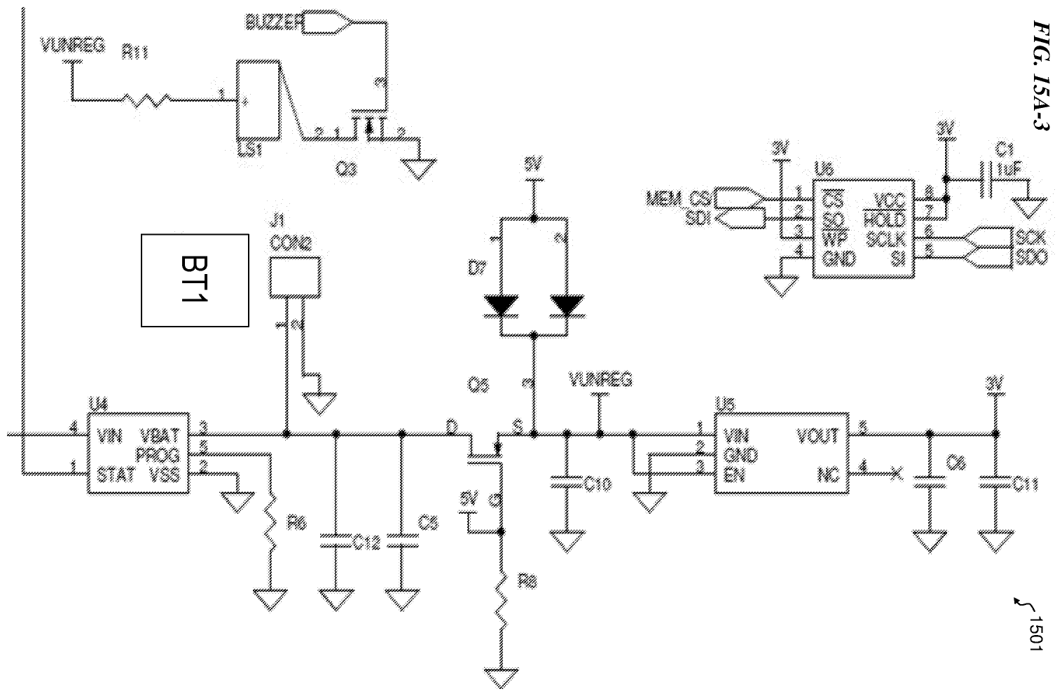

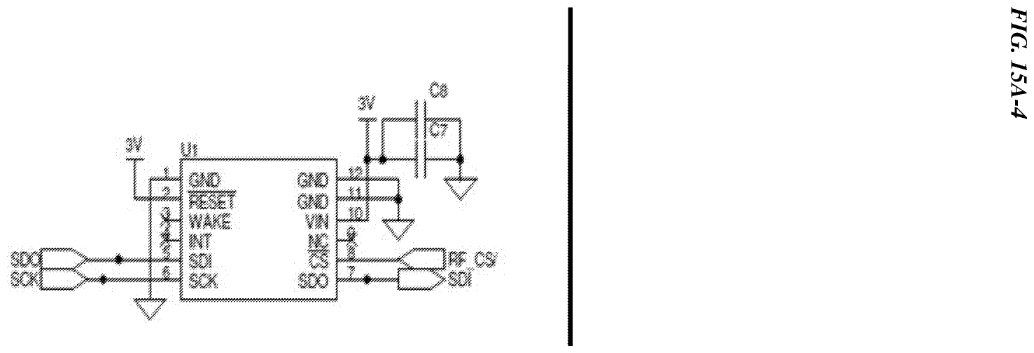

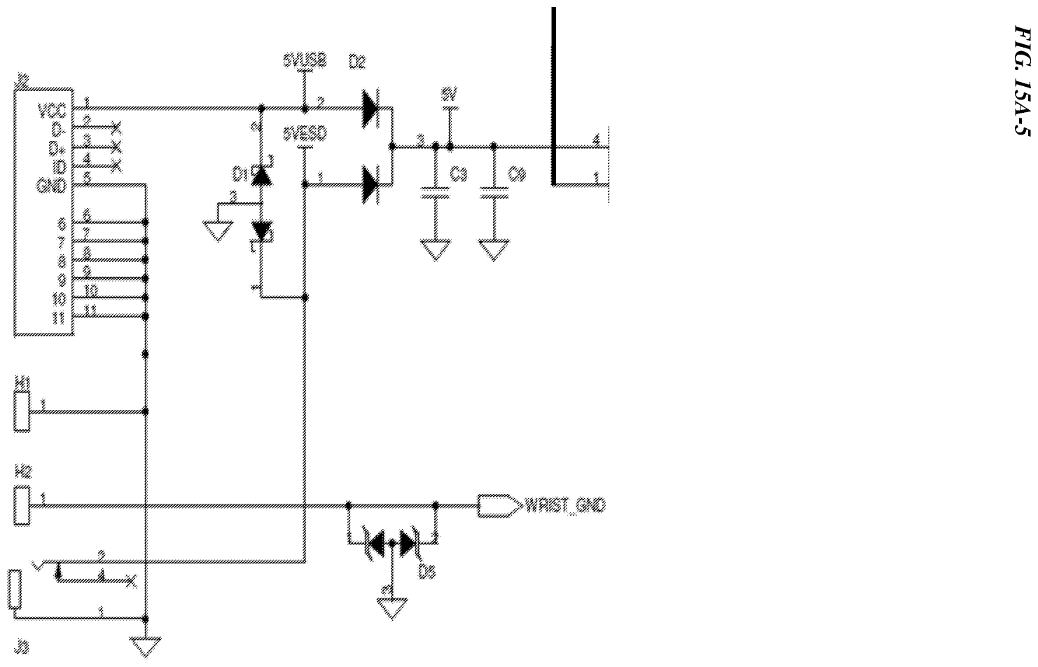

FIG. 15A is a schematic diagram of a sensor unit 1501, according to some embodiments of the present invention. This embodiment includes an LCD touch-screen display for status display and an audible speaker or buzzer to annunciate or alert status.

FIG. 15B is a simplified block diagram of a sensor unit circuit 1502 (e.g., in some embodiments, implemented by the circuit 1501 of FIG. 15A), according to some embodiments of the present invention

In some embodiments, the sensor unit circuit 1502 can receive electrical power from either internal battery 1533 (BT1), which, in some embodiments, includes a rechargeable lithium-ion battery, or from an external 5V supply. Battery 1533 (BT1) interfaces to the circuit of sensor unit circuit 1502 by plugging into connector 1541 (J1, in some embodiments, a connector to which lithium-ion battery 1533 (BT1) connects).

In some embodiments, an external 5V supply 1513 is connected to the sensor unit circuit 1502 via micro-USB connector 1542 (J2, in some embodiments, a micro-USB connector to which an external 5V supply can be connected to charge the sensor unit's lithium-ion battery 1533 and simultaneously supply the sensor unit's electronics). There are many available 5V micro-USB chargers well known to persons of skill in the art and available in the marketplace usable for external 5V supply 1513. This 5V supply 1513 will charge internal battery 1533 (BT1) via lithium-ion battery-charger integrated circuit (IC) 1554 (U4), and will also supply power to the electronics of sensor unit circuit 1502 directly.

In some embodiments, an external 5V supply 1516 (such as one in interface unit 1230) is also connected to the sensor unit via conducting cable connector 1543 (J3, in some embodiments, the connector by which the sensor unit connects to a conducting cable 120 that in turn connects to an interface unit 1230; one conductor of conducting cable 120 connects the sensor unit to earth ground through interface unit 1230. Optionally, a second conductor may supply five volts (5V) to the sensor unit, which both charges the sensor unit's lithium-ion battery and supplies the sensor unit's electronics). In this case, the ring conductor of the conducting cable is at ground, and the tip conductor of the conducting cable is at 5V. This 5V supply 1516 will also charge internal battery 1533 (BT1) via charger IC 1554 (U4, in some embodiments, a battery-charger IC that charges the sensor unit's lithium-ion battery 1533 (BT1) when voltage is supplied to the sensor unit either via its cable connection to the interface unit, or via a connection to its charging connector J2; in some embodiments, U4 is implemented using a Microchip MCP73831T), and will also supply power to the electronics of sensor unit circuit 1502 directly. In some embodiments, a connector 1544 (J4) is provided by which microcontroller 1552 (U2) may be programmed.

In some embodiments, dual diode 1512 (D2) permits an external 5V supply to be connected to both connector 1542 (J2) and connector 1543 (J3); whichever voltage is higher will forward bias its diode and reverse bias the other diode, thereby preventing one 5V supply from back-driving the other. In some embodiments, ESD-protection diodes 1511 and 1515 (D1 and D5) are provided as ESD-protection diodes that prevent ESD shocks (i.e., voltage spikes caused by, e.g., static electricity from contact to a user) received by the sensor unit at charging connector J2, sensor unit cable connector J3, and/or conductive wrist strap contacts H1 and H2, from damaging the sensor unit's electronics. In some embodiments, ESD-protection diodes 1511 and 1515 (D1 and D5) are implemented using NXP Semiconductor's part number PESD3V3U1UT.

In some cases, only the charged battery 1533 provides circuit power. In these cases, the battery 1533 of sensor unit circuit 1502 is likely charged using a conventional external battery charger when not in use. When there is no external 5V supply, battery 1533 (BT1) supplies voltage to voltage regulator 1555 (U5, in some embodiments, a voltage regulator IC that produces a steady 3V output from voltage supplied by battery 1533 (BT1), or voltage supplied by the interface unit via the sensor unit's cable connection 1543 to the interface unit 1230, or voltage supplied by charging connector 1542 (J2); in some embodiments, U5 is implemented using a Microchip MIC5365-3.0YC5), which generates a stable 3V output for the logic circuitry of sensor unit circuit 1502. The voltage from battery 1533 (BT1) is also directly supplied to some analog components, such as the back-light of LCD display 1562.

When an external 5V supply is connected to either connector 1542 (J2) or connector 1543 (J3), this voltage is supplied to regulator IC 1555 (U5) through diode 1517 (D7), which prevents voltage from battery 1533 (BT1) from connecting to the input side of charger IC 1554 (U4) when an external 5V supply is not connected. Since regulator IC 1555 (U5) can be supplied by any of three voltage sources (battery 1533 (BT1), voltage from connector 1542 (J2), or voltage from connector 1543 (J3)), and these three voltage sources are connected in parallel at the input of regulator IC 1555 (U5), diodes 1512 (D2) and 1517 (D7) prevent any of these voltage sources from back-driving the others. P-channel field-effect transistor (FET) 1575 (Q5) prevents the external 5V supply from connecting directly to battery 1533 (BT1), because when an external 5V supply is connected, the gate of FET 1575 (Q5) will be pulled high, thereby disabling conduction through the channel of the FET 1575. Note that, in some embodiments, the diode body (which is not shown) of FET 1575 (Q5) is oriented so that its anode connects to battery 1533 (BT1), thereby preventing conduction from the external 5V supply to battery 1533 (BT1) through the body diode.

In some embodiments, microcontroller 1552 (U2, a microcontroller IC that detects sensor unit contact with the operator's wrist, detects sensor unit connection to an interface unit 1230 via a conducting cable 120 or 170, and manages the sensor unit's memory, RF connection, alarm output, and/or display unit; in some embodiments, microcontroller 1552 is implemented using a Microchip PIC16LF18857) detects sensor unit connection to the operator's wrist, detects sensor unit connection to an interface unit via the conducting cable, and controls the sensor unit's peripherals. In some embodiments, the peripherals include a buzzer 1561, an LCD touch-screen display 1562, and radio-frequency (RF) communication module 1551 (U1, in some embodiments, an RF module that communicates with system monitors and/or interface units; in some embodiments, RF communication module 1551 is implemented using a Microchip MRF24J40MA), which provides an RF data link with system monitors 321 and/or interface units 1230.

In some embodiments, non-volatile memory IC 1556 (U6, in some embodiments, a non-volatile memory IC that records sensor unit connection status information; in some embodiments, non-volatile memory IC 1556 is implemented using an Adesto AT25DF081A-SSH-B) is used to store data, such as sensor unit ID, ESD-protection-state changes, and retains this data when power to the sensor unit circuit 1502 is lost.

In some embodiments, buzzer 1561 (LS1, in some embodiments, a buzzer that produces an audible alarm to alert the human operator when the sensor unit is not providing ESD protection; in some embodiments, buzzer 1561 is implemented using a Soberton ST-0503-3) is controlled by the microcontroller 1552 via FET 1563 (Q3), and is used to generate an audible alarm to alert the operator or user when necessary.

In some embodiments, touch-screen display 1562 (LCD1) is a combination color LCD display unit with back-light, and touch-screen input. In some embodiments, touch-screen display 1562 is an LCD display module with backlight and touch-screen interface that displays status information and permits the operator to configure the sensor unit; in some embodiments, touch-screen display 1562 is implemented using a Newhaven NHD-1.8-128160EF-CTXI#-T. In some embodiments, power to the display-unit logic can be disabled by microcontroller 1552 (U2) via FET 1574 (Q4) to conserve power. Power to the back-light is controlled via FET 1571 (Q1). In some embodiments, an analog voltage is supplied by microcontroller 1552 (U2) to the gate of FET 1571 (Q1); in some embodiments, microcontroller 1552 (U2) can vary this voltage and thereby vary the brightness of the back-light. The touch-screen display 1562 shows ESD connection and safety status, and/or battery level. In some embodiments, touch-screen display 1562 is configured with soft buttons on its touch screen to receive user input and perform functions—in some embodiments, including but not limited to turn power off, reset, and mute audible alarms.

In some embodiments, two wrist contacts 805A and 805B (See FIG. 8) connect to the sensor unit at connectors H1 and H2. In some embodiments, H1 and H2 are contacts that connect to wrist contacts 805A and 805B on the two halves of the sensor unit's conductive wrist strap 115. In some embodiments, H1 grounds contact 805A on one half of the wrist strap, and H2 supplies a voltage to contact 805B on the other half of the wrist strap as part of a system for detecting when both halves of the wrist strap are in contact with an operator's skin. When both wrist contacts 805A and 805B are in contact with an operator's skin, the skin permits electrical current to flow from the 3V supply of sensor unit circuit 1502, through resistors R10 and R12, to connector H2, through the operator's skin, to connector H1, and then to the sensor unit's supply ground 1547. This current will pull a skin-current-path data-input pin (e.g., pin 23) of microcontroller 1552 (U2) to a relatively low voltage level, since resistor R10 is chosen to be much larger than the cumulative resistance of resistor R12 and the expected skin resistance between the two wrist contacts 805A and 805B connected to connectors H1 and H2.

When either of connectors H1 and H2 are no longer in contact with an operator's skin, the current path between connectors H1 and H2 is broken, so current will no longer flow through resistor R12. The skin-current-path data-input pin (e.g., pin 23) of microcontroller 1552 (U2) will therefore be pulled up to the sensor unit's 3V supply by resistor R10, and microcontroller 1552 (U2) will thereby detect that an operator is not properly wearing the sensor unit circuit 1502.

In some embodiments, the two-conductor conducting cable 120 plugs into connector 1543 (J3). The conducting cable 120 (see FIG. 12) may be used in either of two ways: (a) the interface unit 1230 may ground both conductors of the conducting cable 120, or (b) the interface unit 1230 may ground the ring conductor 1548 and supply 5V to the tip conductor 1549.

In the former case, (a), the conducting cable's ring conductor 1548 is connected directly to the sensor unit's ground 1547, and the tip conductor 1549 connects to resistor R9, which then connects to a cable-connection data-input pin (e.g., pin 21) of microcontroller 1552 (U2). When the conducting cable 120 is connected to both the sensor unit (e.g., 810 of FIG. 8) and the interface unit (e.g., 1230 of FIG. 12), then the interface unit 1230 will short-circuit the two conductors together, so cable-connection data-input pin (e.g., pin 21) of microcontroller 1552 (U2) will be pulled to a low voltage level through resistor R9. When, on the other hand, the conducting cable is disconnected from either the sensor unit or the interface unit, the connection between the tip and ring connections on connector 1543 (J3) will be broken, and cable-connection data-input pin (e.g., pin 21) of microcontroller 1552 (U2) will be pulled to an intermediate voltage level by a voltage divider formed by resistors R2 and R7.

In the latter case (b), the conducting cable's ring conductor 1548 is again connected directly to the sensor unit's ground 1547, and the tip conductor 1549 again connects to resistor R9. However, when the conducting cable 120 is connected to the interface unit 1230, the 5V which the interface unit 1230 is supplying to the tip conductor 1549 will cause the cable-connection data-input pin 1521 (e.g., pin 21) of microcontroller 1552 (U2) to be pulled up to a high voltage level. Alternatively, if the conducting cable 120 is disconnected from either the sensor unit 810 or the interface unit 1230, resistors R2 and R7 will again form a voltage divider which pulls the cable-connection data-input pin 1521 (e.g., pin 21) of microcontroller 1552 (U2) to an intermediate voltage level.

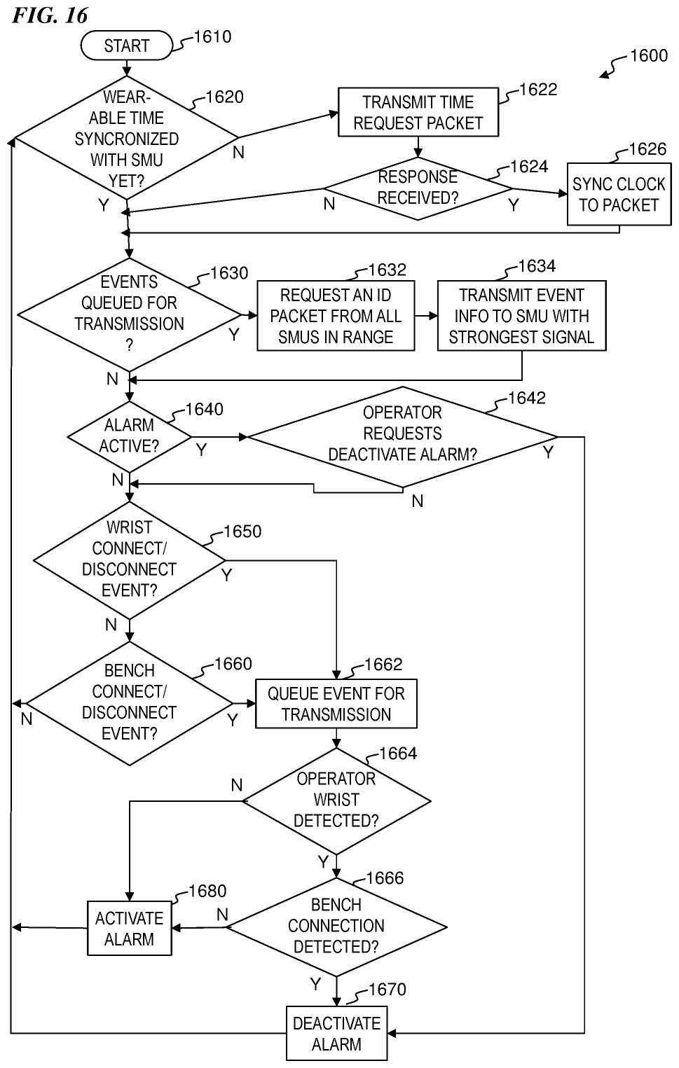

FIG. 16 is a flowchart of a method 1600 usable with various ones of the sensor-unit embodiments described herein, according to some embodiments of the present invention. In some embodiments, the firmware of sensor-unit system 800 executes code executing the method 1600 of the flow chart in FIG. 16. In some embodiments, method 1600 includes starting at start block 1610, which passes control to block 1620; determining at block 1620 whether the time value in the wearable sensor unit 810 is synchronized with the time value in system monitor 321 (also called SMU), and if YES, then passing control to block 1630, and if NO, then passing control to block 1622, in which sensor unit 810 transmits a time-request packet to system monitor 321 and passes control to block 1624. If block 1624 determines YES that a response was received to the time-request packet, control passes to block 1626, which uses the response to synchronize the clock in the sensor unit 810 to the time value in the response, and to correct the time stamps on events recorded prior to reception of the response packet, and control passes to block 1630; but if block 1624 determines NO that no response was received to the time-request packet, then control passes to block 1630 to check and queue events, even though some time inaccuracy may result for some events until control loops back to block 1620 to redetermine whether the time value in the wearable sensor unit 810 is synchronized with the time value in system monitor 321. When control is passed to block 1630, block 1630 determines whether there are events queued for transmission to system monitor 321, and if YES, then block 1632 requests an identifier (ID) packet from all system monitors 321 (SMUs) within communication range, and block 1634 transmits event information to system monitor 321 determined to have the strongest signal, and control passes to block 1640; if block 1630 determines NO there are no events queued for transmission to system monitor 321, control passes to block 1640 from block 1630. Block 1640 determines whether an alarm is active, and if YES then control passes to block 1642 that determines whether an operator (human user) has requested to deactivate the alarm, and if yes, then control passes to block 1670 which deactivates the alarm. If block 1640 determines NO there is no alarm active or block 1642 determines NO there is no operator request to deactivate the alarm, control passes to block 1650 that determines whether there is a wrist (i.e., sensor-unit) connect/disconnect event. If block 1650 determines NO there is no wrist (i.e., sensor-unit) connect/disconnect event then block 1660 determines whether there is a bench (i.e., work-area interface-unit) connect/disconnect event. If block 1660 determines NO there is no bench (i.e., interface-unit) connect/disconnect event then control passes back to block 1620. Else, if either block 1650 determines YES there is a wrist (i.e., sensor-unit) connect/disconnect event or block 1660 determines YES there is a bench (i.e., interface-unit) connect/disconnect event, control passes to block 1662 that queues the event for transmission to system monitor 321 and control passes to block 1664 to check for connection to the operator's wrist. If block 1664 determines NO there is no electrical connection to the operator's wrist control passes to block 1680 that activates the alarm and passes control to block 1620; else, if block 1664 determines YES there is an electrical connection to the operator's wrist, then control passes to block 1666 to check for electrical connection to the bench (interface unit). If block 1666 determines YES there is an electrical connection to the bench (interface unit), then control passes to block 1670 that deactivates the alarm and control passes back to block 1620.

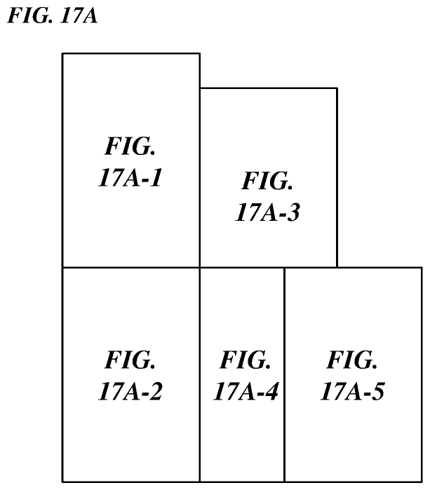

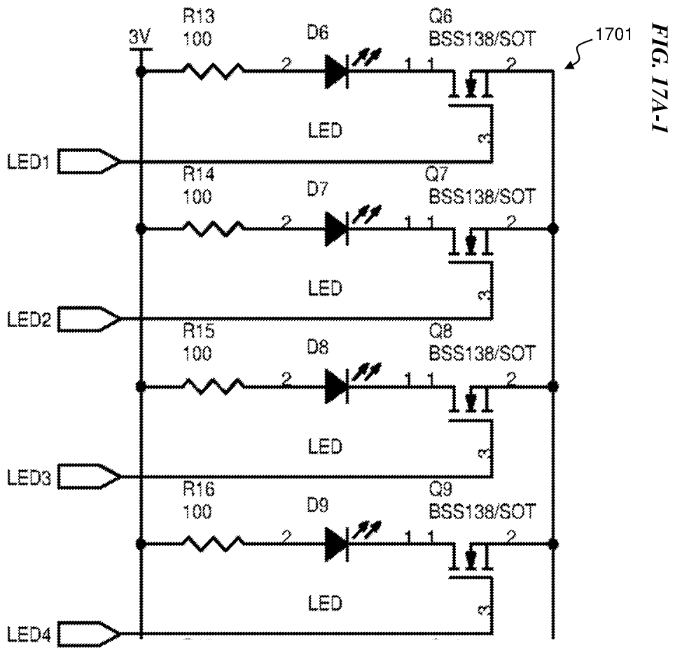

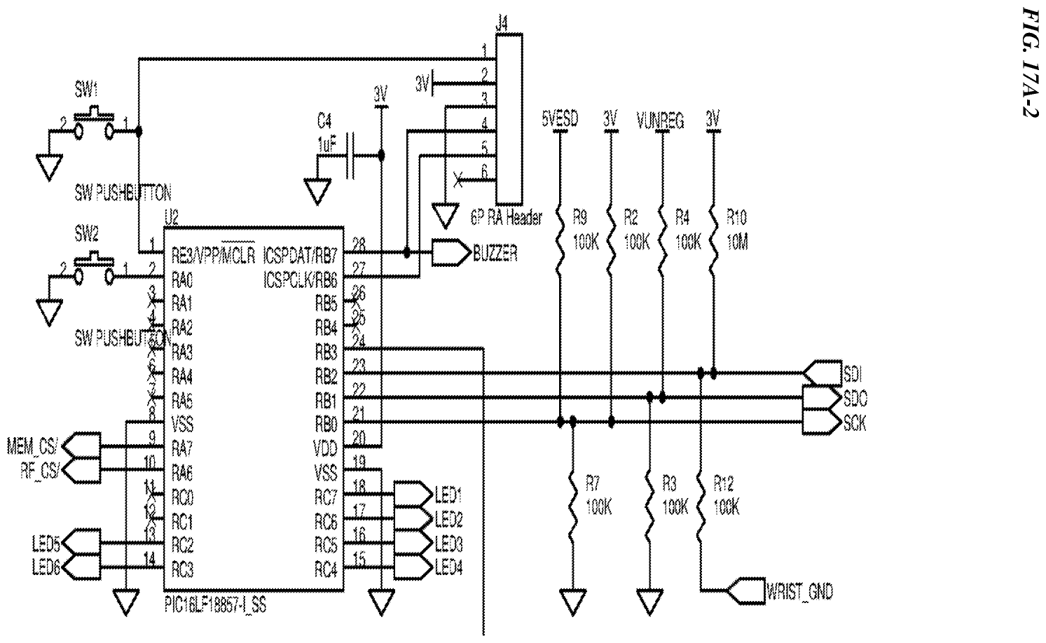

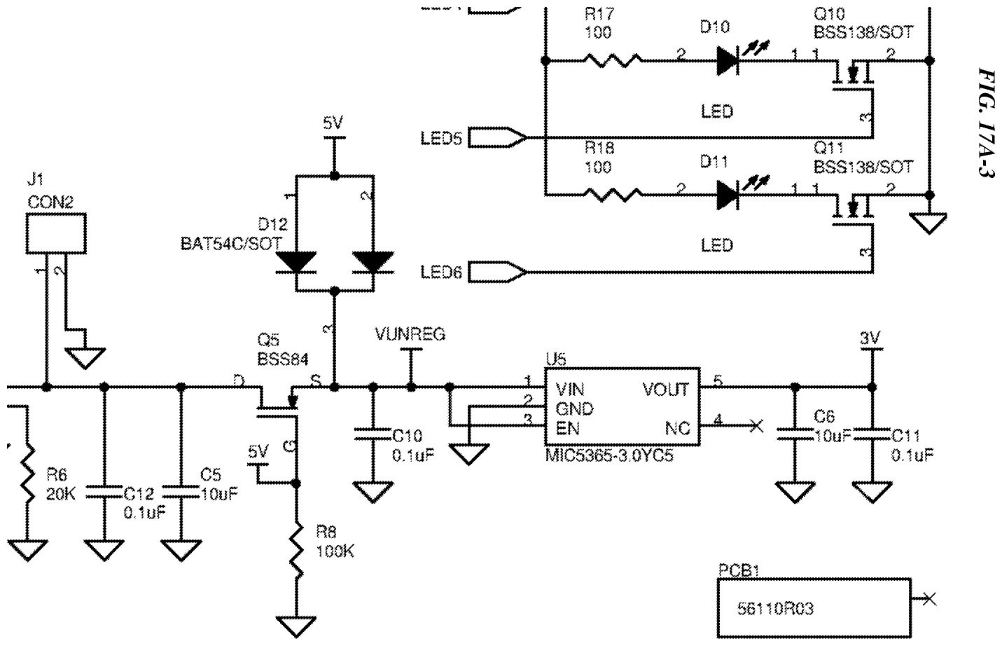

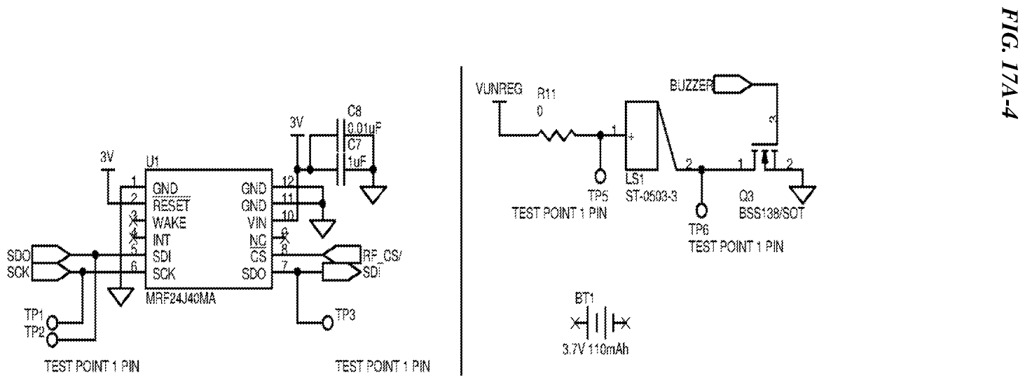

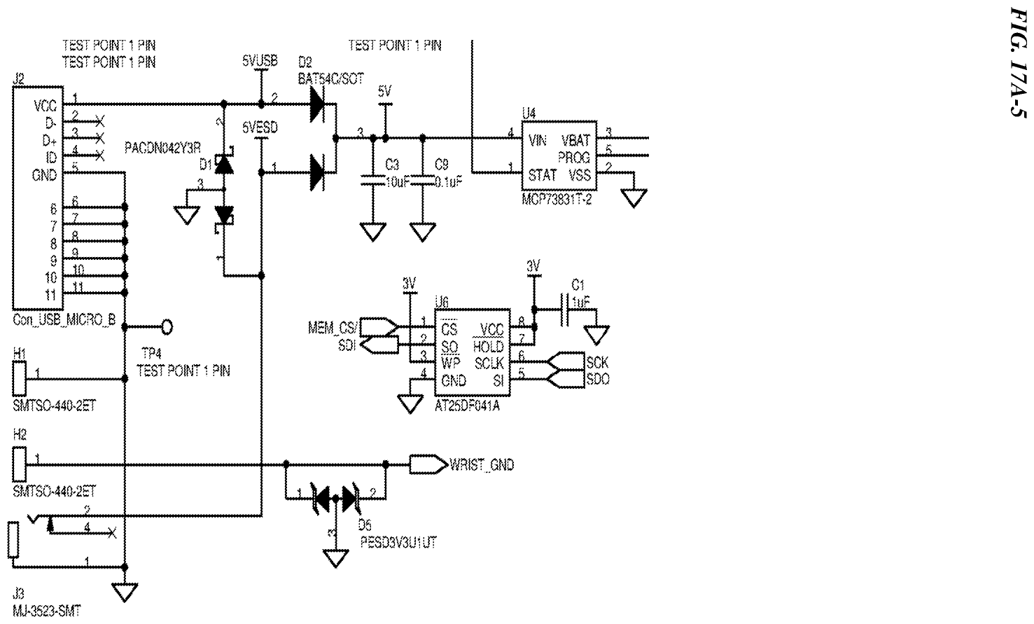

FIG. 17A is a schematic diagram of an alternative sensor unit 1701 that uses LED status indicators and user-input switches in place of the LCD used by sensor unit 1501 of FIG. 15A, according to some embodiments of the present invention. In some embodiments, rather than the touchscreen 1562 (LCD1) of sensor unit 1501 as shown in FIG. 15A, the sensor unit 1701 includes LEDs D6 through D11, which act as visual indicators for the operator, and configuration switches SW1 and SW2, which permit the operator to configure sensor unit 1701. Other aspects of sensor unit 1701 are substantially similar to those of sensor units 1501 and 1502.

FIG. 17B is a block diagram of a sensor unit circuit 1702 that uses LED status indicators, rather than the LCD used by sensor unit 1502 of FIG. 15B, according to some embodiments of the present invention. In some embodiments, rather than the touchscreen 1562 (LCD1) of sensor unit 1502 as shown in FIG. 15B, the sensor unit 1702 includes LEDs D6 through D11, which act as visual indicators for the operator, and configuration switches SW1 and SW2, which permit the operator to configure sensor unit 1702. Other aspects of sensor unit 1702 are substantially similar to those of sensor units 1501 and 1502.

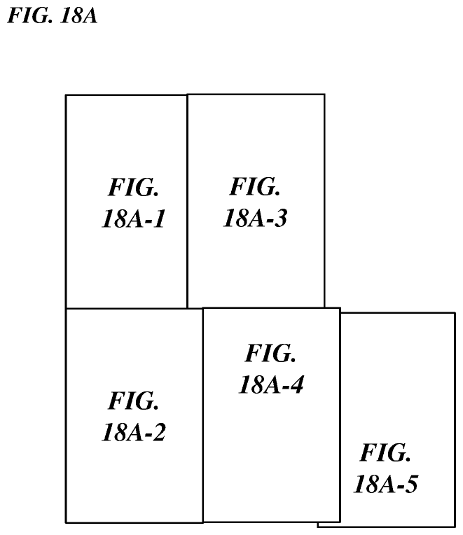

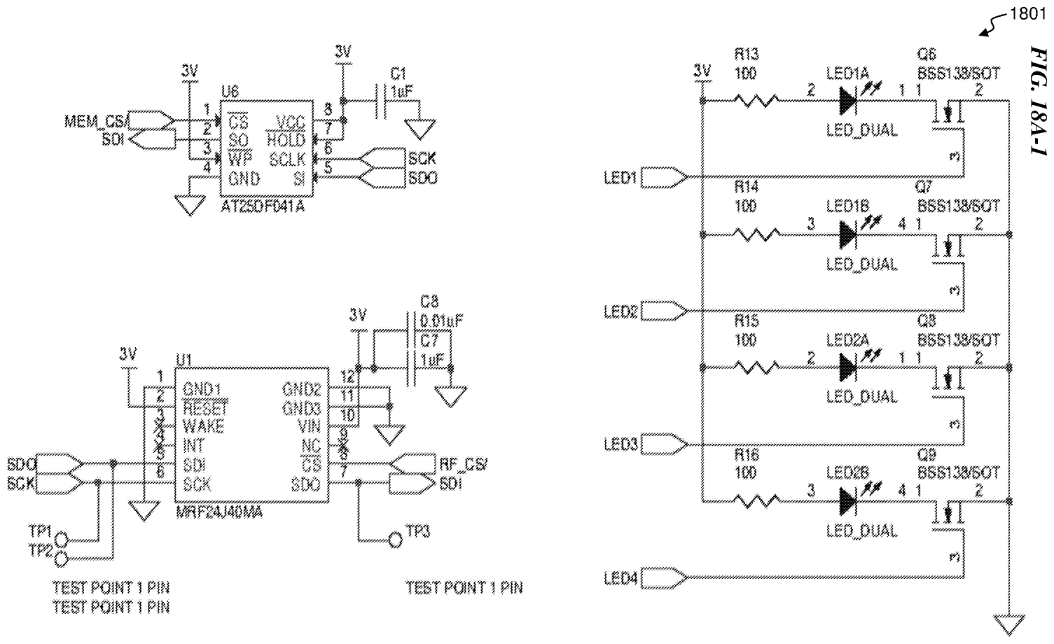

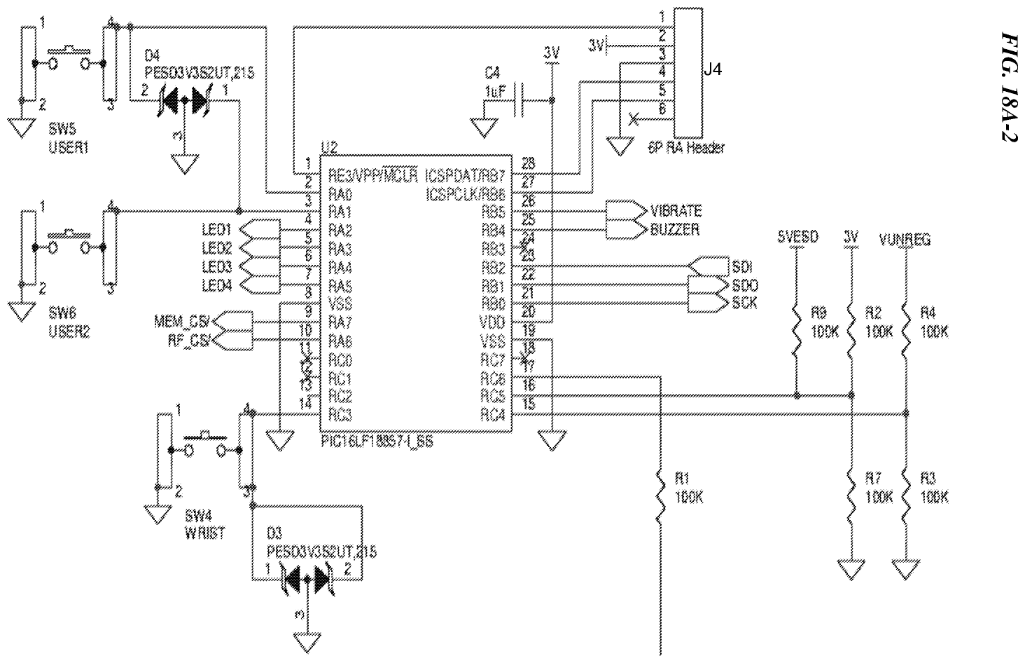

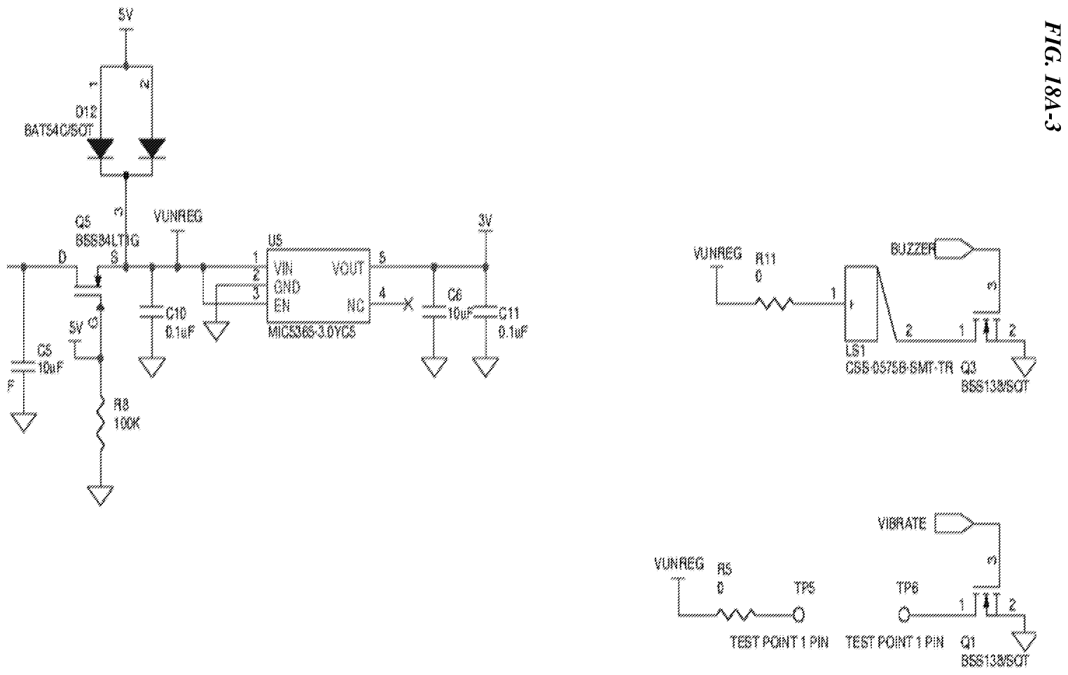

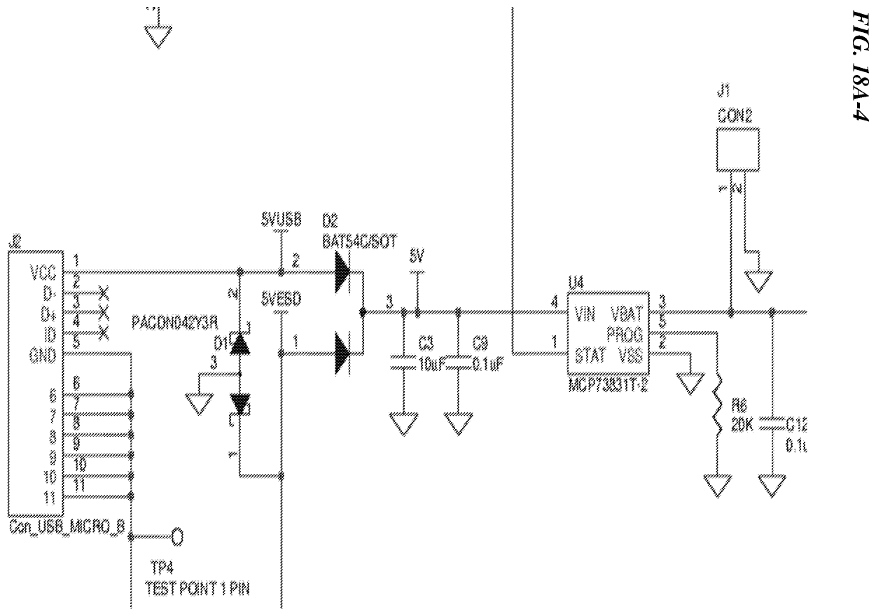

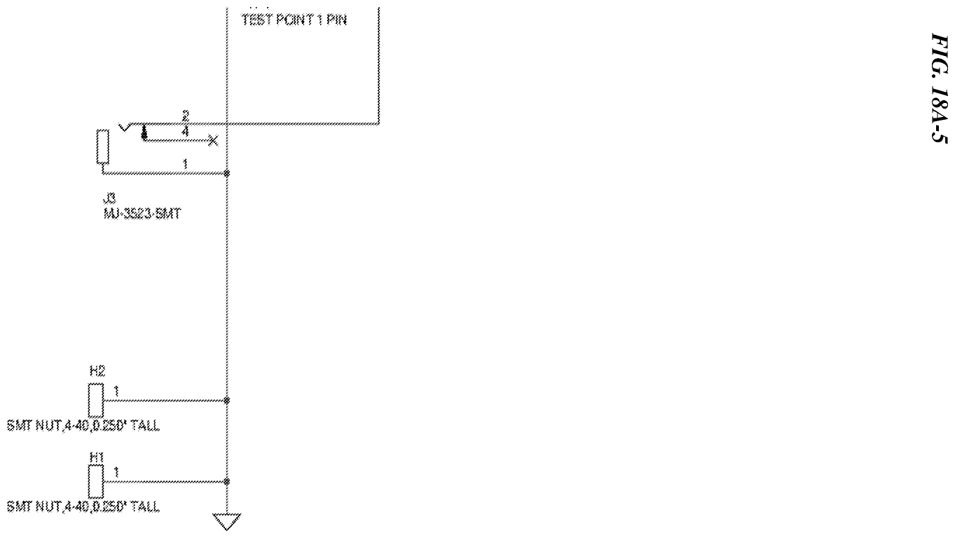

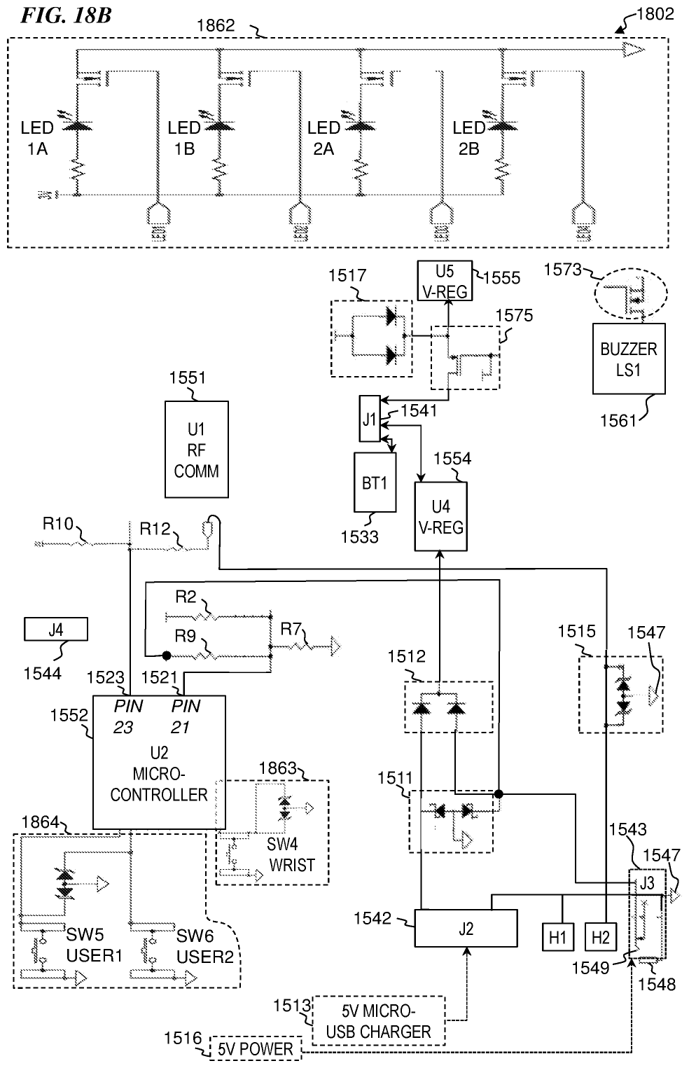

FIG. 18A is a schematic diagram of an alternative sensor unit 1801 that uses LED status indicators and switches, rather than the LCD used by sensor unit 1501 of FIG. 15A, according to some embodiments of the present invention. In some embodiments, rather than the touchscreen 1562 (LCD1) of sensor unit 1501 as shown in FIG. 15A, the sensor unit 1801 includes LEDs LED1A, LED1B, LED2A, and LED2B, which act as visual indicators for the operator, and switches SW5 and SW6, which permit the operator to configure sensor unit 1801. In some embodiments, sensor unit 1801 does not detect the conductivity of the operator's wrist, but instead uses switch SW4 to detect that the sensor unit is strapped to the operator's wrist. In some embodiments, sensor unit 1801 also includes test points TP5 and TP6, where an alternate alarm unit, such as a haptic alarm, may be connected to the circuit and controlled by microcontroller U2. Other aspects of sensor unit 1801 are substantially similar to those of sensor units 1501 and 1502.

FIG. 18B is a block diagram of a sensor unit circuit 1802 that uses LED status indicators, rather than the LCD, and a wrist-strap switch rather than skin-resistance sensing, used by sensor unit 1502 of FIG. 15B, according to some embodiments of the present invention. In some embodiments, rather than the touchscreen 1562 (LCD1) of sensor unit 1501 as shown in FIG. 15A, the sensor unit 1801 includes LEDs LED1A, LED1B, LED2A, and LED2B, which act as visual indicators for the operator, and configuration switches SW5 and SW6, which permit the operator to configure sensor unit 1801. In some embodiments, sensor unit 1802 does not detect the conductivity of the operator's wrist, but instead uses switch SW4 to detect that the sensor unit is strapped to the operator's wrist. In some embodiments, sensor unit 1802 also includes connectors to which an alternate alarm unit, such as a haptic alarm, may be connected to the circuit and controlled by microcontroller 1552. Other aspects of sensor unit 1802 are substantially similar to those of sensor units 1501 and 1502.

In some embodiments, the present invention provides a system that includes: a wearable ESD device configured to be worn by a user, the wearable ESD device having: an electrode that provides electrical conductivity between the wearable ESD device and the user's skin; an electrical connection configured to be connected to a workstation that has a connection to an earth ground, and a wearable-device communications circuit configured to report a plurality of parameters including an indication of an electrical connection between the wearable ESD device and the earth ground at the work station.

In some embodiments, the present invention provides a system that includes: a wearable ESD device configured to be worn by a user, the wearable ESD device having: electronics circuitry that measures electrical conductivity between the wearable ESD device and the user's skin; and a wearable-device communications circuit configured to transmit, to an ESD data-collection station, a plurality of parameters including an indication of the electrical conductivity of the user's skin to an earth ground at a work station.

In some embodiments, the present invention provides a wearable ESD device system that includes: a first wearable ESD device configured to be worn by a user, wherein the first wearable ESD device includes: a machine-readable identification number associated with the first wearable ESD device; an electrical connection configured to be connected to a workstation that has a connection to an earth ground; an electrode that provides electrical conductivity between the wearable ESD device and the user's skin; and a wearable-device communications circuit configured to transmit, to an ESD data-collection station or system monitor, a plurality of parameters including the identification number and an indication of an electrical connection between the user's skin and the earth ground at the work station.

Some embodiments of the wearable ESD device system further include electronics that measures integrity of a connection from the wearable ESD device to earth ground, wherein the communications circuit is further configured to report a parameter representing integrity of a connection from the wearable ESD device to earth ground.

Some embodiments of the wearable ESD device system further include a user-interface output device operably connected to the communications circuit and configured to visually indicate whether the electrical conductivity between the wearable ESD device and the user's skin is acceptable according to criteria of an electrostatic discharge (ESD) policy.

Some embodiments of the wearable ESD device system further include a user-interface output device operably connected to the communications circuit and configured to audibly indicate whether the electrical conductivity between the wearable ESD device and the user's skin is acceptable according to criteria of an electrostatic discharge (ESD) policy.

Some embodiments of the wearable ESD device system further include a user-interface output device operably connected to the communications circuit and configured to indicate by a haptic output whether the electrical conductivity between the wearable ESD device and the user's skin is acceptable according to criteria of an electrostatic discharge (ESD) policy.

Some embodiments of the wearable ESD device system further include a user-interface output device operably connected to the communications circuit and configured to indicate by a visible output indication whether the electrical conductivity between the wearable ESD device and the user's skin is acceptable according to criteria of an electrostatic discharge (ESD) policy.

Some embodiments of the wearable ESD device system further include the ESD data-collection station, wherein the ESD data-collection station is configured to store in a non-volatile storage medium a record of the ESD compliance of a plurality of wearable ESD devices similar to the first wearable ESD device. In some such embodiments, the record is encrypted to protect against tampering with, erasing or changing the record. In some embodiments, the encryption includes a block-chain distributed encryption that is communicated to a plurality of block-chain-encryption storage locations.

Some embodiments of the wearable ESD device system further include an electrostatic discharge (ESD) mat, wherein the ESD mat includes: an electrical interface configured to elicit and receive information from each wearable ESD device that is connected to the ESD mat, and an ESD-mat communications circuit configured to report a plurality of parameters including a value of the electrical conductivity of the user's skin and integrity of a connection from the wearable ESD device to earth ground to an ESD data-collection station.

Some embodiments of the wearable ESD device system further include an electrostatic discharge (ESD) interface unit, wherein the ESD interface unit includes: an electrical interface configured to elicit and receive information from each wearable ESD device that is connected to the ESD interface unit, and an ESD-interface-unit communications circuit configured to report, to the ESD data-collection system monitor, a plurality of parameters including an indication of whether there is an electrical connection between the user's skin and the earth ground at the workstation, and an indication of an integrity of a connection from each respective wearable ESD device to earth ground.

In some embodiments of the wearable ESD device system, the ESD-mat communications circuit communicates wirelessly to the ESD data-collection station.

In some embodiments of the wearable ESD device system, the wearable-device communications circuit communicates wirelessly to the ESD data-collection station.

Some embodiments of the wearable ESD device system further include a user-interface output device operably connected to the communications circuit and configured to alert the user by a user-perceptible indication output whether either the electrical conductivity between the wearable ESD device and the user's skin or the integrity of a connection from the wearable ESD device to earth ground becomes unacceptable according to criteria of an ESD policy.

In some embodiments, the present invention provides a wearable electrostatic discharge (ESD) device method that includes: providing a wearable ESD device configured to be worn by a user; measuring electrical conductivity between the wearable ESD device and the user's skin; and communicating a plurality of parameters from the wearable ESD device including a value of the electrical conductivity of the user's skin to an ESD data-collection station.

Some embodiments of the wearable ESD device method further include: measuring integrity of a connection from the wearable ESD device to earth ground, and communicating to the ESD data-collection station a parameter representing integrity of a connection from the wearable ESD device to earth ground.

Some embodiments of the wearable ESD device method further include: outputting a user-perceptible indication of whether the electrical conductivity between the wearable ESD device and the user's skin is acceptable according to criteria of an electrostatic discharge (ESD) policy.

Some embodiments of the wearable ESD device method further include: collecting, at the ESD data-collection station, and storing in a non-volatile storage medium, a record of the ESD compliance of a plurality of wearable ESD devices. In some such embodiments, the record is encrypted to protect against tampering with, erasing or changing the record.

Some embodiments of the wearable ESD device method further include: providing an electrostatic discharge (ESD) mat having an ESD-mat information processor; eliciting and receiving to the ESD-mat information processor, data from each wearable ESD device that is connected to the ESD mat, and communicating from the ESD-mat, to a central data-collection station such as an ESD data-collection system monitor, a plurality of parameters including a value of the electrical conductivity of the user's skin and integrity of a connection from the wearable ESD device to earth ground.

In some of the wearable ESD device method embodiments, the ESD-mat information processor communicates wirelessly to the central data-collection station.

In some of the wearable ESD device method embodiments, the wearable-device communicates wirelessly to the central data-collection station.

Some embodiments of the wearable ESD device method further include outputting from the wearable ESD device a user-perceptible indication output whether either the electrical conductivity between the wearable ESD device and the user's skin or the integrity of a connection from the wearable ESD device to earth ground becomes unacceptable according to criteria of an electrostatic discharge (ESD) policy.

In some embodiments, the present invention provides a wearable ESD device apparatus that includes: a wearable ESD device configured to be worn by a user; means for measuring electrical conductivity between the wearable ESD device and the user's skin; and means for communicating, to an ESD data-collection station from the wearable ESD device, a plurality of parameters including a value of the electrical conductivity of the user's skin.

In some embodiments, the present invention provides an ESD interface system for monitoring ESD compliance of a plurality of users including a first user at a plurality of work stations including a first work station that has a connection to an earth ground. This ESD interface system includes: a first ESD interface device configured to be associated with the first work station, wherein the first ESD interface device includes: a machine-readable identification number associated with the first ESD interface device; an electrical connection configured to be connected to the earth ground at the first work station; an electrical connection that provides electrical conductivity between the first ESD interface device and a first wearable ESD device, wherein the first wearable ESD device has a machine-readable identification number and is configured to provide electrical contact to the first user's skin; and an ESD-interface-device communications circuit configured to communicate, to a data-collection ESD-compliance system monitor, a plurality of parameters including: the identification number of the first wearable ESD device, the identification number of the first ESD interface device, and an indication of an electrical connection between the user's skin and the earth ground at the first work station.

Some embodiments of the ESD interface system further include circuitry that measures electrical conductivity between the first ESD interface device and the earth ground, wherein the ESD-interface-device communications circuit of the first ESD interface device is further configured to transmit, to the ESD-compliance system monitor, a value of the electrical conductivity between the first interface device and the earth ground.

Some embodiments of the ESD interface system further include a user-interface output device operably connected to the ESD-interface-device communications circuit and configured to audibly indicate whether the electrical conductivity between the first ESD interface device and the user's skin is acceptable according to criteria of an ESD policy.

Some embodiments of the ESD interface system further include a user-interface output device operably connected to the ESD-interface-device communications circuit and configured to indicate by a visible output whether the electrical conductivity between the first ESD interface device and the user's skin is acceptable according to criteria of an electrostatic discharge (ESD) policy.

Some embodiments of the ESD interface system further include the ESD-compliance system monitor, wherein the ESD-compliance system monitor is configured to store in a non-volatile storage medium a record of the ESD compliance of a plurality of wearable ESD devices each having a machine-readable identification number and configured to provide electrical contact to a user's skin. In some such embodiments, the record is encrypted to protect against tampering with, erasing or changing the record.

Some embodiments of the ESD interface system further include the first wearable ESD device, wherein the first wearable ESD device includes: an electrical interface configured to elicit and receive information from each ESD interface device that is connected to the first wearable ESD device, and a wearable-device communications circuit configured to report a plurality of parameters including a value of the electrical conductivity of the user's skin and integrity of a connection from the first wearable ESD device to earth ground to the ESD-compliance system monitor.

In some embodiments of the ESD interface system, the first wearable-device communications circuit communicates wirelessly to the ESD-compliance system monitor.

In some embodiments of the ESD interface system, the first ESD interface device communications circuit communicates wirelessly to the ESD-compliance system monitor.

Some embodiments of the ESD interface system further include a user-interface output device operably connected to the ESD interface device communications circuit and configured to alert the user by a user-perceptible indication output whether either the electrical conductivity between the first wearable ESD device and the first user's skin or the integrity of a connection from the wearable ESD device to earth ground becomes unacceptable according to criteria of an electrostatic discharge (ESD) policy.

Some embodiments of the ESD interface system further include circuitry that measures integrity of a connection from the first ESD interface device to earth ground, wherein the ESD interface device communications circuit is further configured to report at least one parameter representing the integrity of the connection from the first ESD interface device to earth ground.

Some embodiments of the ESD interface system further include a user-interface output device operably connected to the ESD interface device communications circuit and configured to visually indicate whether the electrical conductivity between the first ESD interface device and the user's skin is acceptable according to criteria of an electrostatic discharge (ESD) policy.

In some embodiments, the present invention provides an ESD interface method that includes: providing a first ESD interface device having an ESD-interface-device information processor and having a serial number associated with a first work station; providing a first wearable ESD device configured to be worn by a first user; connecting the first ESD interface device to earth ground at the first work station; connecting the first wearable ESD device to the first ESD interface device; and communicating, to an ESD-compliance system monitor from the ESD interface device, a plurality of parameters including an indication of an electrical connection of the first user's skin to the earth ground.

Some embodiments of the ESD interface method further include determining integrity of a connection from the first ESD interface device to earth ground, and communicating to the ESD-compliance system monitor a parameter representing the integrity of the connection from the first ESD interface device to earth ground.

Some embodiments of the ESD interface method further include outputting a user-perceptible indication of whether the electrical conductivity between the first ESD interface device and the user's skin is acceptable according to criteria of an electrostatic discharge (ESD) policy.

Some embodiments of the ESD interface method further include collecting, at the ESD-compliance system monitor, and storing in a non-volatile storage medium, a record of the ESD compliance of a plurality of ESD interface devices. In some such embodiments, the record is encrypted to protect against tampering with, erasing or changing the record.

Some embodiments of the ESD interface method further include providing an ESD mat; eliciting and receiving to the ESD-interface-device information processor from each wearable ESD device that is connected to the ESD mat, and communicating, from ESD interface device to the ESD-compliance system monitor, a plurality of parameters including a value of the electrical conductivity of the user's skin and integrity of a connection from the respective wearable ESD device to earth ground.

Some embodiments of the ESD interface method further include eliciting and receiving to the ESD-interface-device information processor from each wearable ESD device that is connected to the ESD interface device, and communicating, from ESD interface device to the ESD-compliance system monitor, a plurality of parameters including: timestamped connection and disconnection events between the ESD interface device and each respective one of a plurality of wearable ESD devices and an indication of the electrical connection to the user's skin and integrity of a connection from the respective wearable ESD device to earth ground.

In some embodiments of the ESD interface method, the plurality of parameters is communicated wirelessly to the ESD-compliance system monitor.

In some embodiments of the ESD interface method, the wearable-device communicates wirelessly to the ESD-compliance system monitor.

Some embodiments of the ESD interface method further include outputting from the wearable ESD device a user-perceptible indication output whether either the electrical conductivity between the wearable ESD device and the user's skin or the integrity of a connection from the wearable ESD device to earth ground becomes unacceptable according to criteria of an electrostatic discharge (ESD) policy.

In some embodiments, the present invention provides an ESD interface apparatus that includes: a first ESD interface device; means for measuring electrical conductivity between the first ESD interface device and an earth ground; and means for communicating a plurality of parameters from the first ESD interface device including a value of the electrical conductivity between the first ESD interface device and an earth ground to an ESD-compliance system monitor. Some such embodiments further include a first wearable ESD device configured to be worn by a first user and having a machine-readable wearable-device number associated with the first wearable ESD device, wherein the first ESD interface device includes means for receiving the machine-readable wearable-device number from the first wearable ESD device, and wherein the plurality of parameters includes the machine-readable wearable-device number from the first wearable ESD device.

In some embodiments, the present invention provides an electrostatic-discharge (ESD) interface system configured to monitor compliance of a plurality of users including a first user at a plurality of work stations including a first work station that has a connection to an earth ground. This ESD interface system includes: a first ESD interface device configured to be associated with the first work station, wherein the first ESD interface device includes: a machine-readable identification number associated with the first ESD interface device; an electrical connection configured to be connected to the earth ground at the first work station; an electrical connection that provides electrical conductivity between the first ESD interface device and a first wearable device, wherein the first wearable device has a machine-readable identification number and is configured to provide electrical contact to the first user's skin; and an ESD-interface-device communications circuit configured to communicate, to a data-collection ESD-compliance system monitor, a plurality of parameters including: the identification number of the first ESD wearable device, the identification number of the first ESD interface device, and an indication of an electrical connection between the user's skin and the earth ground at the first work station.

Some embodiments of the ESD interface system further include: circuitry that measures electrical conductivity between the first ESD interface device and the earth ground, wherein the ESD-interface-device communications circuit of the first ESD interface device is further configured to transmit, to the ESD-compliance system monitor, a value of the electrical conductivity between the first interface device and the earth ground.

Some embodiments of the ESD interface system further include: a user-interface output device operably connected to the ESD-interface-device communications circuit and configured to audibly indicate whether the electrical conductivity between the first ESD interface device and the user's skin is acceptable according to criteria of an ESD policy.

In some embodiments of the ESD interface system, the first ESD interface device further includes: a user-interface output device operably connected to the ESD-interface-device communications circuit and configured to indicate by a visible output whether the electrical conductivity between the first ESD interface device and the user's skin is acceptable according to criteria of an ESD policy.

Some embodiments of the ESD interface system further include: the ESD-compliance system monitor, wherein the ESD-compliance system monitor is configured to store in a non-volatile storage medium a record of the ESD compliance of a plurality of wearable devices each having a machine-readable identification number and configured to provide electrical contact to a user's skin. In some such embodiments, the record is encrypted to protect against tampering with, erasing or changing the record.

Some embodiments of the ESD interface system further include: the first wearable device, wherein the first wearable device includes: an electrical interface configured to elicit and receive information from each ESD interface device that is connected to the wearable device, and a wearable-device communications circuit configured to report a plurality of parameters including a value of the electrical conductivity of the user's skin and integrity of a connection from the wearable device to earth ground to a ESD-compliance system monitor. In some such embodiments, the first wearable-device communications circuit communicates wirelessly to the ESD-compliance system monitor.

In some embodiments of the ESD interface system, the first ESD interface device communications circuit communicates wirelessly to the ESD-compliance system monitor.

In some embodiments of the ESD interface system, the first ESD interface device further includes a user-interface output device operably connected to the ESD interface device communications circuit and configured to alert the user by a user-perceptible indication output whether either the electrical conductivity between the first wearable device and the first user's skin or the integrity of a connection from the wearable device to earth ground becomes unacceptable according to criteria of an ESD policy.

In some embodiments of the ESD interface system, the first ESD interface device further includes circuitry that measures integrity of a connection from the first ESD interface device to earth ground, wherein the ESD interface device communications circuit is further configured to report at least one parameter representing the integrity of the connection from the first ESD interface device to earth ground.

In some embodiments of the ESD interface system, the first ESD interface device further includes a user-interface output device operably connected to the ESD interface device communications circuit and configured to visually indicate whether the electrical conductivity between the first ESD interface device and the user's skin is acceptable according to criteria of an ESD policy.

In some embodiments, the present invention provides an electrostatic-discharge (ESD) interface method that includes: providing a first ESD interface device having an ESD-interface-device information processor and having a serial number associated with a first work station; providing a first wearable device configured to be worn by a first user; connecting the first ESD interface device to earth ground at the first work station; connecting the first wearable device to the first ESD interface device; and communicating, to a ESD-compliance system monitor from the ESD interface device, a plurality of parameters including an indication of an electrical connection of the first user's skin to the earth ground.

Some embodiments of the ESD interface method further include: determining integrity of a connection from the first ESD interface device to earth ground, and communicating to the ESD-compliance system monitor a parameter representing integrity of the connection from the first ESD interface device to earth ground.

Some embodiments of the ESD interface method further include: outputting a user perceptible indication of whether the electrical conductivity between the first ESD interface device and the user's skin is acceptable according to criteria of an ESD policy.

Some embodiments of the ESD interface method further include: collecting, at the ESD-compliance system monitor, and storing in a non-volatile storage medium, a record of the ESD compliance of a plurality of ESD interface devices. In some such embodiments, the record is encrypted to protect against tampering with, erasing or changing the record.

Some embodiments of the ESD interface method further include: providing an ESD mat; eliciting and receiving to the ESD-interface-device information processor from each wearable device that is connected to the ESD mat, and communicating, from ESD interface device to the ESD-compliance system monitor, to report a plurality of parameters including a value of the electrical conductivity of the user's skin and integrity of a connection from the wearable device to earth ground.

In some embodiments of the ESD-interface method, the plurality of parameters is communicated by the first ESD interface device wirelessly to the ESD-compliance system monitor.

In some embodiments of the ESD-interface method, the wearable-device communicates wirelessly to the ESD-compliance system monitor.

Some embodiments of the ESD interface method further include: outputting from the wearable device a user-perceptible indication output whether either the electrical conductivity between the wearable device and the user's skin or the integrity of a connection from the wearable device to earth ground becomes unacceptable according to criteria of an ESD policy.

In some embodiments, the present invention provides an electrostatic-discharge (ESD) compliance-monitoring system configured to monitor ESD compliance of a plurality of users at a plurality of work stations that each has a connection to an earth ground, wherein each respective user of the plurality of users is associated with a respective one of a plurality of wearable ESD devices, wherein each respective wearable ESD device has an associated wearable-device identification code, wherein each respective work station of the plurality of work stations is associated with a respective one of a plurality of ESD interface devices, wherein each respective ESD interface device has an associated interface-device identification code, and wherein the ESD-compliance-monitoring system includes: a first ESD system monitor, wherein the first ESD system monitor is configured to receive communications from at least one device of the plurality of wearable ESD devices and the plurality of ESD interface devices, wherein the communications include the identification code associated with a respective one of the plurality of wearable ESD devices and the identification code associated with a respective one of the ESD interface devices to which the respective one of the plurality of wearable ESD devices becomes connected, and wherein the first ESD system monitor is programmed to record connection and disconnection events between the respective ones of the plurality of wearable ESD devices and the respective ones of the ESD interface devices to which the respective ones of the plurality of wearable ESD devices are connected and to record associated timestamps for each of the connection and disconnection events.

In some embodiments of the ESD-compliance-monitoring system, each respective one of the plurality of ESD interface devices includes a user-proximity detector 1344 that detects disconnected-user events associated with a detected presence of a user at its respective work station who has not connected their wearable ESD device to the respective ESD interface device within a preset amount of time, and the communications include indications of such disconnected-user events, and the first ESD system monitor is programmed to record the disconnected-user events and to record associated timestamps for each of the disconnected-user events.

Some embodiments of the ESD-compliance-monitoring system further include the plurality of wearable ESD devices; and the plurality of ESD interface devices.

Some embodiments of the ESD-compliance-monitoring system further include a plurality of ESD system monitors, wherein the plurality of ESD system monitors includes the first ESD system monitor and at least one additional ESD system monitor; and a computer server configured to receive and aggregate data from each of the plurality of ESD system monitors and to generate aggregate reports of ESD compliance for the plurality of ESD system monitors.

In some embodiments of the ESD-compliance-monitoring system, each respective one of the plurality of ESD interface devices includes a user-interface that elicits and receives job identifiers from users, the communications include the job identifiers, and the first ESD system monitor is programmed to record the job identifiers and associated timestamps for times between each pair of the connection and disconnection events.

In some embodiments of the ESD-compliance-monitoring system, each respective one of the plurality of ESD interface devices includes a user-interface that elicits and receives part identifiers associated with parts at each respective work station, the communications include the part identifiers, and the first ESD system monitor is programmed to record the part identifiers and associated timestamps for times between each pair of the connection and disconnection events.

In some embodiments of the ESD-compliance-monitoring system, each respective one of the plurality of ESD interface devices includes a user-interface that elicits and receives part serial numbers associated with parts at each respective work station, the communications include the part serial numbers, and the first ESD system monitor is programmed to record the part serial numbers and associated timestamps for times between each pair of the connection and disconnection events.

In some embodiments of the ESD-compliance-monitoring system, the first ESD system monitor is programmed to encrypt the recorded connection and disconnection events and associated timestamps.

In some embodiments of the ESD-compliance-monitoring system, each respective one of the plurality of ESD interface devices includes a user-interface that elicits and receives job identifiers from users, each respective one of the plurality of ESD interface devices includes a user-interface that receives part identifiers associated with parts at each respective work station, the communications include the job identifiers and the part identifiers, and the first ESD system monitor is programmed to record the connection and disconnection events, and the job identifiers and the part identifiers and associated timestamps for times between each pair of the connection and disconnection events. In some other such embodiments, the first ESD system monitor is programmed to encrypt and record the connection and disconnection events, and the job identifiers and the part identifiers and associated timestamps for times between each pair of the connection and disconnection events.

Some embodiments of the ESD-compliance-monitoring system further include the plurality of wearable ESD devices; the plurality of ESD interface devices; a plurality of ESD system monitors, wherein the plurality of ESD system monitors includes the first ESD system monitor and at least one additional ESD system monitor; and a computer server configured to receive and aggregate data from each of the plurality of ESD system monitors and to generate aggregate reports of ESD compliance for the plurality of ESD system monitors; wherein each respective one of the plurality of ESD interface devices includes a user-interface that elicits and receives billing identifiers from users, wherein the communications include the billing identifiers, and wherein the server is programmed to record the billing identifiers and associated timestamps for times between each pair of the connection and disconnection events for each one of the plurality of users.

In some embodiments, the present invention provides an electrostatic-discharge (ESD) compliance-monitoring method for monitoring ESD compliance of a plurality of users at a plurality of work stations that each has a connection to an earth ground, wherein each respective user of the plurality of users is associated with a respective one of a plurality of wearable ESD devices, wherein each respective wearable ESD device has an associated wearable-device identification code, and wherein each respective work station of the plurality of work stations is associated with a respective one of a plurality of ESD interface devices, wherein each respective ESD interface device has an associated interface-device identification code. This ESD-compliance-monitoring method includes: receiving communications from at least one device of the plurality of wearable ESD devices and the plurality of ESD interface devices, wherein the communications include the identification code associated with a respective one of the plurality of wearable ESD devices and the identification code associated with a respective one of the ESD interface devices to which the respective one of the plurality of wearable ESD devices becomes connected, and recording connection and disconnection events between the respective ones of the plurality of wearable ESD devices and the respective ones of the ESD interface devices to which the respective ones of the plurality of wearable ESD devices are connected and recording associated timestamps for each of the connection and disconnection events.

Some embodiments of the ESD-compliance-monitoring method further include: detecting disconnected-user events associated with a detected presence of a user at a respective one of the plurality of work stations who has not connected their wearable ESD device to the respective ESD interface device within a preset amount of time, wherein the receiving communications include receiving indications of such disconnected-user events, and recording the disconnected-user events and associated timestamps for each of the disconnected-user events.

Some embodiments of the ESD-compliance-monitoring method further include: providing a plurality of ESD system monitors each configured to receive communications from at least one of the plurality of wearable ESD devices and the plurality of ESD interface devices, wherein the communications include the identification code associated with a respective one of the plurality of wearable ESD devices and the identification code associated with a respective one of the ESD interface devices to which the respective one of the plurality of wearable ESD devices becomes connected; receiving and aggregating data into a computer server from each of the plurality of ESD system monitors; and generating, by the computer server, aggregate reports of ESD compliance for the plurality of ESD system monitors.

In some embodiments of the ESD-compliance-monitoring method, each respective one of the plurality of ESD interface devices includes a user-interface that elicits and receives job identifiers from users, the communications include the job identifiers, and the method further includes recording the job identifiers and associated timestamps for times between each pair of the connection and disconnection events.

In some embodiments of the ESD-compliance-monitoring method, each respective one of the plurality of ESD interface devices includes a user-interface that elicits and receives part identifiers associated with parts at each respective work station, the communications include the part identifiers, and the recording the part identifiers and associated timestamps for times between each pair of the connection and disconnection events.

In some embodiments of the ESD-compliance-monitoring method, each respective one of the plurality of ESD interface devices includes a user-interface that elicits and receives part serial numbers associated with parts at each respective work station, the communications include the part serial numbers, and the method further includes recording the part serial numbers and associated timestamps for times between each pair of the connection and disconnection events.

Some embodiments of the ESD-compliance-monitoring method further include, for each respective one of the plurality of users, recording times and durations of when each respective user's wearable device was connected to which ones of the plurality of ESD interface devices for timekeeping purposes.

In some embodiments of the ESD-compliance-monitoring method, the first ESD system monitor is programmed to encrypt the recorded connection and disconnection events and associated timestamps.

In some embodiments of the ESD-compliance-monitoring method, each respective one of the plurality of ESD interface devices includes a user-interface that elicits and receives job identifiers from users, each respective one of the plurality of ESD interface devices includes a user-interface that receives part identifiers associated with parts at each respective work station, the communications include the job identifiers and the part identifiers, and the method further includes recording the connection and disconnection events, and the job identifiers and the part identifiers and associated timestamps for times between each pair of the connection and disconnection events. In some such embodiments, the method further includes encrypting and recording the connection and disconnection events, and the job identifiers and the part identifiers and associated timestamps for times between each pair of the connection and disconnection events.

In some embodiments, the present invention provides an electrostatic-discharge (ESD) compliance-monitoring system for monitoring ESD compliance of a plurality of users at a plurality of work stations that each has a connection to an earth ground. This ESD-compliance-monitoring system includes: a plurality of wearable ESD devices, wherein each respective user of the plurality of users is associated with a respective one of the plurality of wearable ESD devices, wherein each respective wearable ESD device has an associated wearable-device identification code; a plurality of ESD interface devices, wherein each respective work station of the plurality of work stations is associated with a respective one of the plurality of ESD interface devices, wherein each respective ESD interface device has an associated interface-device identification code. This ESD-compliance-monitoring system includes: means for receiving communications from at least one device of the plurality of wearable ESD devices and the plurality of ESD interface devices, wherein the communications include the identification code associated with a respective one of the plurality of wearable ESD devices and the identification code associated with a respective one of the ESD interface devices to which the respective one of the plurality of wearable ESD devices becomes connected, and means for recording connection and disconnection events between the respective ones of the plurality of wearable ESD devices and the respective ones of the ESD interface devices to which the respective ones of the plurality of wearable ESD devices are connected and recording associated timestamps for each of the connection and disconnection events.

In some embodiments, the present invention provides an apparatus that includes a wearable electrostatic discharge (ESD) device configured to be worn by a user; means for measuring electrical conductivity between the wearable ESD device and the user's skin; and means for communicating, to an ESD data-collection system monitor, a plurality of parameters from the wearable ESD device including a value of the electrical conductivity of the user's skin.

In some embodiments, the present invention provides an ESD-interface apparatus that includes: a first ESD interface device; means for measuring electrical conductivity between the first ESD interface device and an earth ground; and means for communicating a plurality of parameters from the first ESD interface device including a value of the electrical conductivity between the first ESD interface device and an earth ground to a ESD-compliance system monitor. Some embodiments of this ESD-interface apparatus further include a first wearable device configured to be worn by a first user and having a machine-readable wearable-device number associated with the first wearable device, wherein the first ESD interface device includes means for receiving the machine-readable wearable-device number from the first wearable device, and wherein the plurality of parameters includes the machine-readable wearable-device number from the first wearable device.

It is to be understood that the above description is intended to be illustrative, and not restrictive. Although numerous characteristics and advantages of various embodiments as described herein have been set forth in the foregoing description, together with details of the structure and function of various embodiments, many other embodiments and changes to details will be apparent to those of skill in the art upon reviewing the above description. The scope of the invention should be, therefore, determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

1.-66. (canceled)

67. A method comprising:
providing a first wearable electrostatic discharge (ESD) device configured to be worn by a user;
assigning a machine-readable identification number to the first wearable device;
determining, by the first wearable ESD device, whether there is an electrical connection between the first wearable ESD device and the user's skin;
communicating, to an ESD data-collection system monitor, a plurality of parameters from the first wearable ESD device including the machine-readable identification number and an indication of the electrical connection to the user's skin; and
outputting a user-perceptible indication of whether the electrical conductivity between the first wearable ESD device and the user's skin is acceptable according to criteria of an electrostatic discharge (ESD) policy.

68. The method of claim 67, further comprising:
measuring integrity of a connection from the first wearable ESD device to earth ground, and
communicating, to the ESD data-collection system monitor, a parameter representing integrity of a connection from the first wearable ESD device to earth ground.

69. The method of claim 67, further comprising:
collecting, at the ESD data-collection system monitor, and storing in a storage medium, a record of ESD compliance of a plurality of wearable ESD devices.

70. The method of claim 67, further comprising:
providing an ESD interface unit having an ESD-interface-unit information processor;
eliciting and receiving to the ESD-interface-unit information processor from each wearable ESD device that is connected to the ESD interface unit, and
communicating from the ESD-interface-unit information processor to report a plurality of parameters including an indication of an electrical connection between the user's skin and the earth ground at the workstation and integrity of a connection from each respective wearable ESD device to earth ground to the ESD data-collection system monitor.

71. The method of claim 70, wherein the ESD-interface-unit information processor communicates wirelessly to the ESD data-collection system monitor.

72. The method of claim 67, wherein the wearable ESD device communicates wirelessly to the ESD data-collection system monitor.

73. The method of claim 67, further comprising:
outputting from the first wearable ESD device a user-perceptible indication output whether either the electrical conductivity between the first wearable ESD device and the user's skin or the integrity of a connection from the first wearable ESD device to earth ground becomes unacceptable according to criteria of an ESD policy.

74. A system comprising:
a first wearable electrostatic discharge (ESD) device configured to be worn by a user and having:
a machine-readable identification number associated with the first wearable ESD device;
an electrical connection configured to be connected to a workstation that has a connection to an earth ground;
an electrode that provides electrical conductivity between the wearable ESD device and the user's skin;
a wrist-contact sensor that detects whether the first wearable ESD device is in contact with the user's skin;
a wearable-device communications circuit configured to transmit, to an ESD data-collection system monitor, a plurality of parameters including the identification number and an indication of an electrical connection between the user's skin and the earth ground at the work station; and
electronics circuitry that measures electrical conductivity between the first wearable ESD device and the user's skin, wherein the wearable-device communications circuit is further configured to transmit, to the ESD data-collection system monitor, a value of the electrical conductivity between the user's skin and the first wearable ESD device.

75. The system of claim 74, the first wearable ESD device further comprising:
a strap; and
a sensor that determines whether the strap has sufficient tautness to provide suitable connection against the user's skin, wherein the wearable-device communications circuit is further configured to transmit, to the ESD data-collection system monitor, an indication of the suitable connection of the user's skin.

76. The system of claim 74, the first wearable ESD device further comprising a user-interface output device operably connected to the communications circuit and configured to audibly indicate whether the electrical conductivity between the wearable ESD device and the user's skin is acceptable according to criteria of an electrostatic discharge (ESD) policy.

77. The system of claim 74, the first wearable ESD device further comprising a user-interface output device operably connected to the communications circuit and configured to indicate by a haptic output indication whether the electrical conductivity between the wearable ESD device and the user's skin is acceptable according to criteria of an electrostatic discharge (ESD) policy.

78. The system of claim 74, further comprising the ESD data-collection system monitor, wherein the ESD data-collection system monitor is configured to store in a storage medium a record of the ESD compliance of a plurality of wearable ESD devices.

79. The system of claim 74, further comprising an electrostatic discharge (ESD) interface unit, wherein the ESD interface unit includes:
an electrical interface configured to elicit and receive information from each wearable ESD device that is connected to the ESD interface unit, and
an ESD-interface-unit communications circuit configured to report, to the ESD data-collection system monitor, a plurality of parameters including an indication of an electrical connection between the user's skin and the earth ground at the workstation, and an indication of integrity of a connection from each respective wearable ESD device to earth ground.

80. The system of claim 79, wherein the ESD-interface-unit communications circuit communicates wirelessly to the ESD data-collection system monitor.

81. The system of claim 74, wherein the wearable-device communications circuit communicates wirelessly to the ESD data-collection system monitor.

82. The system of claim 74, the first wearable ESD device further comprising a user-interface output device operably connected to the communications circuit and configured to alert the user by a user-perceptible indication output whether either an electrical connection between the first wearable ESD device and the user's skin or the integrity of a connection from the first wearable ESD device to earth ground becomes unacceptable according to criteria of an electrostatic discharge (ESD) policy.

83. The system of claim 74, the first wearable ESD device further comprising:
electronics that measures integrity of a connection from the first wearable ESD device to earth ground, wherein the communications circuit is further configured to report a parameter representing integrity of a connection from the first wearable ESD device to earth ground.

84. The system of claim 74, the first wearable ESD device further comprising a user-interface output device operably connected to the communications circuit and configured to visually indicate whether the electrical conductivity between the first wearable ESD device and the user's skin is acceptable according to criteria of an electrostatic discharge (ESD) policy.

85. A system comprising:
a first wearable electrostatic discharge (ESD) device configured to be worn by a user and having:
a machine-readable identification number associated with the first wearable ESD device;
an electrical connection configured to be connected to a workstation that has a connection to an earth ground;
an electrode that provides electrical conductivity between the wearable ESD device and the user's skin;
a sensor that detects whether the first wearable ESD device is in contact with the user's skin;
a wearable-device communications circuit configured to transmit, to an ESD data-collection system monitor, a plurality of parameters including the identification number and an indication of an electrical connection between the user's skin and the earth ground at the work station; and
a user-interface output device operably connected to the communications circuit and configured to output a user-perceptible indication of whether the electrical conductivity between the wearable ESD device and the user's skin is acceptable according to criteria of an electrostatic discharge (ESD) policy.

86. A system comprising:
a first wearable electrostatic discharge (ESD) device configured to be worn by a user and having:
a machine-readable identification number associated with the first wearable ESD device;
an electrical connection configured to be connected to a workstation that has a connection to an earth ground;
an electrode that provides electrical conductivity between the wearable ESD device and the user's skin;
a wearable-device communications circuit configured to transmit, to an ESD data-collection system monitor, a plurality of parameters including the identification number and an indication of an electrical connection between the user's skin and the earth ground at the work station; and
a user-interface output device operably connected to the communications circuit and configured to indicate whether the user is grounded.

87. An electrostatic-discharge (ESD) interface system configured to monitor compliance of a plurality of users including a first user at a plurality of work stations including a first work station that has a connection to an earth ground, the system comprising:
a first ESD interface device configured to be associated with the first work station, wherein the first ESD interface device includes:
a machine-readable identification number associated with the first ESD interface device;
an electrical connection configured to be connected to the earth ground at the first work station;
an electrical connection that provides electrical conductivity between the first ESD interface device and a first wearable device, wherein the first wearable device has a machine-readable identification number and is configured to provide electrical contact to the first user's skin;
an ESD-interface-device communications circuit configured to communicate, to a data-collection ESD-compliance system monitor, a plurality of parameters including:
the identification number of the first ESD wearable device,
the identification number of the first ESD interface device, and
an indication of an electrical connection between the user's skin and the earth ground at the first work station; and
a user-interface output device operably connected to the ESD-interface-device communications circuit and configured to provide a first user-perceptible indication of whether the electrical conductivity between the first ESD interface device and the user's skin is acceptable according to criteria of an ESD policy.

88. The system of claim 87, the first ESD interface device further comprising:
circuitry that measures electrical conductivity between the first ESD interface device and the earth ground, wherein the ESD-interface-device communications circuit of the first ESD interface device is further configured to transmit, to the ESD-compliance system monitor, a value of the electrical conductivity between the first interface device and the earth ground.

89. The system of claim 87, wherein the first user-perceptible indication includes an audible output.

90. The system of claim 87, wherein the first user-perceptible indication includes a visible output.

91. The system of claim 87, further comprising the ESD-compliance system monitor, wherein the ESD-compliance system monitor is configured to store in a storage medium a record of the ESD compliance of a plurality of wearable devices each having a machine-readable identification number and configured to provide electrical contact to a user's skin.

92. The system of claim 87, further comprising the ESD-compliance system monitor, wherein the ESD-compliance system monitor is configured to store in a storage medium a record of the ESD compliance of a plurality of wearable devices each having a machine-readable identification number and configured to provide electrical contact to a user's skin, wherein the record is encrypted to protect against tampering with, erasing or changing the record.

93. The system of claim 87, further comprising the first wearable device, wherein the first wearable device includes:
an electrical interface configured to elicit and receive information from each ESD interface device that is connected to the wearable device, and
a wearable-device communications circuit configured to report a plurality of parameters including a value of the electrical conductivity of the user's skin and an indication of a connection integrity from the wearable device to earth ground to a ESD-compliance system monitor.

94. The system of claim 93, wherein the first wearable-device communications circuit communicates wirelessly to the ESD-compliance system monitor.

95. The system of claim 87, wherein the first ESD interface device communications circuit communicates wirelessly to the ESD-compliance system monitor.

96. The system of claim 87, wherein the user-interface output device is further configured to provide a second user-perceptible indication of whether the integrity of a connection from the wearable device to earth ground becomes unacceptable according to criteria of an ESD policy.

97. The system of claim 87, wherein the first ESD interface device further includes circuitry that measures integrity of a connection from the first ESD interface device to earth ground, wherein the ESD interface device communications circuit is further configured to report at least one parameter representing the integrity of the connection from the first ESD interface device to earth ground.

98. The system of claim 87, wherein the first user-perceptible indication includes a haptic output.

99. A system comprising:
a first wearable electrostatic discharge (ESD) device configured to be worn by a user and having:
a machine-readable identification number associated with the first wearable ESD device;
an electrical connection configured to be connected to a workstation that has a connection to an earth ground;
an electrode that provides electrical conductivity between the wearable ESD device and the user's skin;
a wearable-device communications circuit configured to transmit, to an ESD data-collection system monitor, a plurality of parameters including the identification number and an indication of an electrical connection between the user's skin and the earth ground at the work station; and
a user-interface output device operably connected to the communications circuit and configured to provide a user-perceptible indication of whether the electrical conductivity between the first wearable ESD device and the user's skin is acceptable according to criteria of an ESD policy.

100. An electrostatic-discharge (ESD) interface method comprising:
providing a first ESD interface device having an ESD-interface-device information processor and having a serial number associated with a first work station;
providing a first wearable device configured to be worn by a first user;
detecting whether the first wearable device is in contact with the user's skin;
connecting the first ESD interface device to earth ground at the first work station;
connecting the first wearable device to the first ESD interface device;
communicating, to a ESD-compliance system monitor from the ESD interface device, a plurality of parameters including an indication of an electrical connection of the first user's skin to the earth ground; and
outputting a user perceptible indication of whether the electrical conductivity between the first ESD interface device and the user's skin is acceptable according to criteria of an ESD policy.

101. The ESD interface method of claim 100, further comprising:
determining integrity of a connection from the first ESD interface device to earth ground, and
communicating to the ESD-compliance system monitor a parameter representing integrity of the connection from the first ESD interface device to earth ground.

102. The ESD interface method of claim 100, further comprising:
collecting, at the ESD-compliance system monitor, and storing in a storage medium, a record of the ESD compliance of a plurality of ESD interface devices.

103. The ESD interface method of claim 100, further comprising:
providing an ESD mat;
eliciting and receiving to the ESD-interface-device information processor from each wearable device that is connected to the ESD mat, and
communicating, from ESD interface device to the ESD-compliance system monitor, to report a plurality of parameters including a value of the electrical conductivity of the user's skin and integrity of a connection from the wearable device to earth ground.

104. The ESD-interface method of claim 103, wherein the plurality of parameters is communicated by the first ESD interface device wirelessly to the ESD-compliance system monitor.

105. The ESD-interface method of claim 100, wherein the wearable-device communicates wirelessly to the ESD-compliance system monitor.

106. The ESD-interface method of claim 100, further comprising:
outputting from the wearable device a user-perceptible indication output whether either the electrical conductivity between the wearable device and the user's skin or the integrity of a connection from the wearable device to earth ground becomes unacceptable according to criteria of an ESD policy.

107. An apparatus comprising:
a wearable electrostatic discharge (ESD) device configured to be worn by a user;
means for measuring electrical conductivity between the wearable ESD device and the user's skin; and
means for communicating, to an ESD data-collection system monitor, a plurality of parameters from the wearable ESD device including a value of the electrical conductivity of the user's skin.

108. An ESD-interface apparatus comprising:
a first ESD interface device;
means for measuring electrical conductivity between the first ESD interface device and an earth ground; and
means for communicating a plurality of parameters from the first ESD interface device including a value of the electrical conductivity between the first ESD interface device and an earth ground to a ESD-compliance system monitor.

109. The apparatus of claim 108, further comprising:
a first wearable device configured to be worn by a first user and having a machine-readable wearable-device number associated with the first wearable device, wherein the first ESD interface device includes means for receiving the machine-readable wearable-device number from the first wearable device, and wherein the plurality of parameters includes the machine-readable wearable-device number from the first wearable device.
